(19) United States
(12) Patent Application Publication
(10) Pub. No.: US 2020/0225213 A1
(43) Pub. Date: Jul. 16, 2020

(54) COMPOSITIONS AND METHODS FOR DETECTING CARDIOTOXICITY

(71) Applicant: AgeX Therapeutics, Inc., Alameda, CA (US)

(72) Inventors: Michael D. West, Mill Valley, CA (US); Jeffrey Janus, Alameda, CA (US)

(73) Assignee: AgeX Therapeutics, Inc., Alameda, CA (US)

(21) Appl. No.: 16/736,802

(22) Filed: Jan. 7, 2020

Related U.S. Application Data
(60) Provisional application No. 62/789,486, filed on Jan. 7, 2019.

(51) Int. Cl. G01N 33/50; C12N 5/077

(52) U.S. Cl. CPC G01N 33/5014; G01N 33/5061; C12N 2535/10; C12N 2506/03; C12N 2533/52; C12N 5/0657

(57) ABSTRACT

A method of screening a composition for cardiotoxicity comprising contacting the composition with cardiomyocytes that have increased fatty acid oxidation and/or diminished glucose oxidation. The cardiomyocytes are preferably prepared by overexpression of COX7A1. The cardiomyocytes are preferably provided in a micropatterned co-culture to provide a mature functional hPSC-CM cardiotoxicity model.

### CROSS REFERENCE TO RELATED APPLICATIONS

This application claims priority to U.S. Provisional Application 62/789,486, filed Jan. 7, 2019, the contents of which are hereby incorporated by reference in its entirety.

### FIELD OF THE INVENTION

This disclosure relates to inventive compositions and methods for detecting cardiotoxicity.

### BACKGROUND

Billions of dollars are invested yearly by the pharmaceutical industry to develop new drugs that are safe and effective. During pharmaceutical development, 30% of drug candidates are rejected due to safety concerns, the majority of which are related to potential cardiotoxic effects (Magdy). Even with existing methods of early drug screening, drugs exhibiting cardiotoxic effects are often rejected late in the development process, with 16% of marketed drugs being withdrawn due to cardiovascular toxicity (Siramshetty).

Existing models to screen for cardiac toxicities have many weaknesses In vivo models, such as rodents and zebrafish, have differing structure, electrophysiology and morphology of cardiomyocytes (CM) relative to human physiology and are well known to constitute a serious limitation for measuring risk of drug-caused arrhythmias in humans (Gintant). Cell line models that overexpress ion channels are known to be inaccurate in detecting human cardiac drug responses and have low sensitivity and specificity (Magdy).

For example, the Harmonization of Technical Requirements for Pharmaceuticals for Human Use guidelines (ICH S7B) for testing of a drug's potential to delay ventricular repolarization measures the drug's effect on cells transfected to express the voltage-gated Kv11.1 potassium channel. The sensitivity and specificity of this assay is 64-82% and 75-88% respectively.

Animal models are problematic because of their substantial physiological differences from humans. Animal models are well known to have limitations for measuring risk of drug-caused arrhythmias in humans. Direct comparison between cardiomyocytes isolated from human pluripotent stem cells (hPSC) or dog and rabbit heart showed the human cells more accurately predicted moxifloxacin induced cardiotoxicity.

Human pluripotent stem cell derived cardiomyocytes (hPSC-CM) including those cardiomyocytes (CM) derived from human Embryonic Stem Cells (hESC-CM), show promise in the early detection of human cardiac drug responses. However, hPSC-CMs used in current in vitro models have deficiencies that limit their use in drug screening. The developmental immaturity of hPSC-CM is a primary limitation of these models for cardiotoxicity screening of drugs, and each improvement made to hPSC-CM maturity will further increase utility (Denning). Deficiencies further include 1) hPSC-CM display morphologic and functional characteristics that are embryonic or fetal-like and not mature thereby hindering the measurement of cardiotoxicities that may affect mature adult heart tissue; 2) the hPSC-CMs are heterogenous and unstructured in vitro, consisting of a mixture of ventricular, atrial and nodal (pacemaker) cell types, making it difficult to recapitulate normal heart function and isolate cardiotoxicities that affect specific cardiac tissue types; 3) deficiencies in hiPSC-CM models relative to hESC-CM models have been reported, for example, robust hypertrophic responses to phenylephrine are recorded in hESC-CM models but not hiPSC-CM models. This difference remains when fibroblasts derived from the hESC are reprogrammed to hiPSC and hIPSC-CMs are subsequently derived from the fibroblasts.

Progress has been made in the field to identify modifications to current hPSC-CM in vitro cardiotoxicity models that help to alleviate these deficiencies, but these deficiencies remain.

### SUMMARY OF THE INVENTION

The invention is directed to methods and compositions for improved screening of compositions for cardiotoxicity. In particular, the methods and compositions of the invention relate to improved screening of pharmaceutical compositions.

In a preferred embodiment, the invention is directed to a method of screening a composition for cardiotoxicity wherein an assay uses cardiomyocytes that have increased fatty acid oxidation and increased oxidative phosphorylation, and/or diminished glucose oxidation.

In a preferred embodiment, the invention is directed to a method of screening a pharmaceutical composition for cardiotoxicity wherein an assay uses cardiomyocytes that primarily utilize fatty acids as an energy source.

In another embodiment according to the invention, is a cardiomyocyte or population of cardiomyocytes that have increased fatty acid oxidation and increased oxidative phosphorylation, and/or diminished glucose oxidation. That cardiomyocyte or population of cardiomyocytes is preferably prepared by overexpression of COX7A1.

In a preferred embodiment, the invention is directed to a cardiomyocyte or population of cardiomyocytes that primarily utilize fatty acids as an energy source. In a further preferred embodiment of the invention, the cardiomyocytes do not utilize glucose as a primary energy source. That cardiomyocyte or population of cardiomyocytes is preferably prepared by overexpression of COX7A1.

In a preferred embodiment of the invention, COX7A1 overexpression is used to metabolically mature the cardiomyocytes to be used in the methods and compositions according to the invention. In a further preferred embodiment, the cardiomyocytes are human pluripotent stem cell-derived cardiomyocytes. In a further preferred embodiment, the cardiomyocytes are mature to the point that they have an established mitochondrial system and engagement of oxidative metabolism.

Another aspect of the invention is a method for generating a mature cardiomyocyte or population of cardiomyocytes, comprising transfecting a human pluripotent stem cell line including but not limited to a human embryonic stem cell line or human induced pluripotent stem cell line with COX7A1, differentiating those cells to produce cardiomyocytes, thereby producing cardiomyocytes via expression of COX7A1. In a preferred embodiment, the mature cardiomyocyte or population of cardiomyocytes have increased fatty acid oxidation and/or diminished glucose oxidation relative to cardiomyocytes differentiated from a human embryonic stem cell line not transfected with COX7A1 or a transfected cell line where COX7A1 expression is not induced. The mature cardiomyocytes in the compositions and methods according to the invention preferably have an increased expression in one or more of CSQ, PLN, RYR2, SERCA/ATP2A2, MyH7, TNNI3, and ADRA1A and/or a decreased expression of one or more of MYH6 and TNNI1 than uninduced cells.

In a further preferred embodiment, COX7A1 is transfected by a knock-in or random integration into the genome of the pluripotent stem cells or in myocardial progenitors such as with an inducible COX7A1 expression cassette. The COX7A1 gene may be inducible via any known inducing agents, such as an antibiotic including, for example, tetracycline. Alternatively, COX7A1 is constitutively expressed.

Another embodiment according to the invention is a method of screening a composition for cardiotoxicity effects, comprising contacting cardiomyocytes according to the invention with the composition and observing the cardiotoxicity effects.

In another embodiment is a micropatterned co-cultured human-pluripotent cell-based in vitro cardiotoxicity assay comprising the cardiomyocytes according to the invention. In a further preferred embodiment, the cardiomyocytes are cocultured with diverse types of cardiac cells including sinus node atrial cells to provide pacing function in vitro, or endothelial cells and/or vascular pericytes. Preferably said co-cultured cells are also modified according to the present invention to express COX7A1. Preferably, the co-culture enhances the structure and/or function of the cardiomyocytes and/or provides a more accurate model of normal adult myocardium.

The present invention also applies to all other somatic cell types as well such as skeletal, muscle, brown adipocytes, as well as other stromal and parenchymal cell types.

### BRIEF DESCRIPTION OF DRAWINGS

For a fuller understanding of the nature and advantages of the present invention, reference should be had to the following detailed description taken in connection with the accompanying drawings.

FIG. 1 depicts results of an anti-COX7A1 Western blot.

FIG. 2 depicts quantitative RT-PCT results wherein COX7A1 mRNA expression level has been normalized to GAPDH; error bar STDEV (n=3).

FIG. 3 depicts a COX7A1 lentiviral vector.

FIG. 4 depicts results of Illumina bead array gene expression analysis, wherein gene expression (shown in relative fluorescence units) is essentially undetectable in iPSC-CM, then increases in expression during fetal development in vivo, then increases further upon reaching adulthood.

FIG. 5 depicts a cross-section of a micropatterned well for a cardiotoxicity assay according to the invention.

### DETAILED DESCRIPTION

Abbreviations

AC—Adult-derived cells

ASC—Adult stem cells

cGMP—Current Good Manufacturing Processes

CM—Cardiomyocytes

DMEM—Dulbecco's modified Eagle's medium

DMSO—Dimethyl sulfoxide

DNAm—Changes in the methylation of DNA that provide a marker or “clock” of the age of cells and tissue.

DNN—Deep Neural Network

DPBS—Dulbecco's Phosphate Buffered Saline

ED Cells—Embryo-derived cells; hED cells are human ED cells

EDTA—Ethylenediamine tetraacetic acid

EFT—Embryonic-Fetal Transition

EG Cells—Embryonic germ cells; hEG cells are human EG cells

EP—Embryonic progenitors

ES Cells—Embryonic stem cells; hES cells are human ES cells

ESC—Embryonic Stem Cells

FACS—Fluorescence activated cell sorting

FBS—Fetal bovine serum

FPKM—Fragments Per Kilobase of transcript per Million mapped reads from RNA sequencing.

GFP—Green fluorescent protein

GMP—Good Manufacturing Practices

HAEC—Human Aortic Endothelial Cell

hED Cells—Human embryo-derived cells

hEG Cells—“Human embryonic germ cells” are stem cells derived from the primordial germ cells of fetal tissue.

hESC—Human Embryonic Stem Cells

hESC-CM—Human pluripotent stem cell-derived cardiomyocytes

hiPS Cells—“Human induced pluripotent stem cells” are cells with properties similar to hES cells obtained from somatic cells after exposure to hES-specific transcription factors such as SOX2, KLF4, OCT4, MYC, or NANOG, LIN28, OCT4, and SOX2.

hPSC—Human pluripotent stem cells is an inclusive term for OCT4-expressing pluripotential cells such as hESCs, hiPSCs, parthenogenically-derived pluripotent stem cells, hEG cells and may include naïve and primed versions of these cell types.

hPSC-CM—Human pluripotent stem cell-derived cardiomyocytes

iPS Cells—“Induced pluripotent stem cells” are cells with properties similar to hES cells obtained from somatic cells after exposure to ES-specific transcription factors such as SOX2, KLF4, OCT4, MYC, or NANOG, LIN28, OCT4, and SOX2, SOX2, KLF4, OCT4, MYC, and (LIN28A or LIN28B), or other combinations of OCT4, SOX2, KLF4, NANOG, ESRRB, NR5A2, CEBPA, MYC, LIN28A and LIN28B.

iTM—Induced Tissue Maturation

iTR—Induced Tissue Regeneration

MEM—Minimal essential medium

MSC—Mesenchymal stem cell

NT—Nuclear Transfer

PBS—Phosphate buffered saline

PPT—“Prenatal-Postnatal Transition” refers to the molecular alterations that occur in cells of placental mammals at or within a week of birth.

RFU—Relative Fluorescence Units

RNA-seq—RNA sequencing

SFM—Serum-Free Medium

St. Dev.—Standard Deviation

TR—Tissue Regeneration

### Definitions

The term “analytical reprogramming technology” refers to a variety of methods to reprogram the pattern of gene expression of a somatic cell to that of a more pluripotent state, such as that of an iPS, ES, ED, EC or EG cell, wherein the reprogramming occurs in multiple and discrete steps and does not rely simply on the transfer of a somatic cell into an oocyte and the activation of that oocyte (see U.S. application nos. 60/332,510, filed Nov. 26, 2001; Ser. No. 10/304,020, filed Nov. 26, 2002; PCT application no. PCT/US02/37899, filed Nov. 26, 2003; U.S. application No. 60/705,625, filed Aug. 3, 2005; U.S. application No. 60/729,173, filed Aug. 20, 2005; U.S. application No. 60/818,813, filed Jul. 5, 2006, PCT/US06/30632, filed Aug. 3, 2006, the disclosure of each of which is incorporated by reference herein).

The term “blastomere/morula cells” refers to blastomere or morula cells in a mammalian embryo or blastomere or morula cells cultured in vitro with or without additional cells including differentiated derivatives of those cells.

The term “cell expressing gene X”, “gene X is expressed in a cell” (or cell population), or equivalents thereof, means that analysis of the cell using a specific assay platform provided a positive result. The converse is also true (i.e., by a cell not expressing gene X, or equivalents, is meant that analysis of the cell using a specific assay platform provided a negative result). Thus, any gene expression result described herein is tied to the specific probe or probes employed in the assay platform (or platforms) for the gene indicated.

The term “cell line” refers to a mortal or immortal population of cells that is capable of propagation and expansion in vitro.

The term “clonal” refers to a population of cells obtained the expansion of a single cell into a population of cells all derived from that original single cells and not containing other cells.

The term “differentiated cells” when used in reference to cells made by methods of this invention from pluripotent stem cells refer to cells having reduced potential to differentiate when compared to the parent pluripotent stem cells. The differentiated cells of this invention comprise cells that could differentiate further (i.e., they may not be terminally differentiated).

The term “embryonic” or “embryonic stages of development” refers to prenatal stages of development of cells, tissues or animals, specifically, the embryonic phases of development of cells compared to fetal and adult cells. In the case of the human species, the transition from embryonic to fetal development occurs at about 8 weeks of prenatal development, in mouse it occurs on or about 16 days, and in the rat species, at approximately 17.5 days post coitum. (http://php.med.unsw.edu.au/embryology/index.php?title=Mouse_Timeline_Detailed).

The term “embryonic stem cells” (ES cells) refers to cells derived from the inner cell mass of blastocysts, blastomeres, or morulae that have been serially passaged as cell lines while maintaining an undifferentiated state (e.g. expressing TERT, OCT4, and SSEA and TRA antigens specific for ES cells of the species). The ES cells may be derived from fertilization of an egg cell with sperm or DNA, nuclear transfer, parthenogenesis, or by means to generate hES cells with hemizygosity or homozygosity in the MHC region. While ES cells have historically been defined as cells capable of differentiating into all of the somatic cell types as well as germ line when transplanted into a preimplantation embryo, candidate ES cultures from many species, including human, have a more flattened appearance in culture and typically do not contribute to germ line differentiation, and are therefore called “ES-like cells.” It is commonly believed that human ES cells are in reality “ES-like”, however, in this application we will use the term ES cells to refer to both ES and ES-like cell lines.

The term “global modulator of TR” or “global modulator of iTR” refers to agents capable of modulating a multiplicity of iTR genes or iTM genes including, but not limited to, agents capable of downregulating COX7A1 while simultaneously up-regulating PCDHB2, or downregulating NAALADL1 while simultaneously up-regulating AMH in cells derived from fetal or adult sources and are capable of inducing a pattern of gene expression leading to increased scarless tissue regeneration in response to tissue damage or degenerative disease.

The term “human embryo-derived” (“hED”) cells refers to blastomere-derived cells, morula-derived cells, blastocyst-derived cells including those of the inner cell mass, embryonic shield, or epiblast, or other totipotent or pluripotent stem cells of the early embryo, including primitive endoderm, ectoderm, mesoderm, and neural crest and their derivatives up to a state of differentiation correlating to the equivalent of the first eight weeks of normal human development, but excluding cells derived from hES cells that have been passaged as cell lines (see, e.g., U.S. Pat. Nos. 7,582,479; 7,217,569; 6,887,706; 6,602,711; 6,280,718; and U.S. Pat. No. 5,843,780 to Thomson). The hED cells may be derived from preimplantation embryos produced by fertilization of an egg cell with sperm or DNA, nuclear transfer, or chromatin transfer, an egg cell induced to form a parthenote through parthenogenesis, analytical reprogramming technology, or by means to generate hES cells with hemizygosity or homozygosity in the HLA region. The term “human embryonic germ cells” (hEG cells) refer to pluripotent stem cells derived from the primordial germ cells of fetal tissue or maturing or mature germ cells such as oocytes and spermatogonial cells, that can differentiate into various tissues in the body. The hEG cells may also be derived from pluripotent stem cells produced by gynogenetic or androgenetic means, i.e., methods wherein the pluripotent cells are derived from oocytes containing only DNA of male or female origin and therefore will comprise all female-derived or male-derived DNA.

The term “human embryonic stem cells” (hES cells) refers to human ES cells.

The term “human induced pluripotent stem cells” refers to cells with properties similar to hES cells, including the ability to form all three germ layers when transplanted into immunocompromised mice wherein said iPS cells are derived from cells of varied somatic cell lineages following exposure to de-differentiation factors, for example hES cell-specific transcription factor combinations: KLF4, SOX2, MYC; OCT4 or SOX2, OCT4, NANOG, and LIN28; or various combinations of OCT4, SOX2, KLF4, NANOG, ESRRB, NR5A2, CEBPA, MYC, LIN28A and LIN28B or other methods that induce somatic cells to attain a pluripotent stem cell state with properties similar to hES cells. However, the reprogramming of somatic cells by somatic cell nuclear transfer (SCNT) are typically referred to as NT-ES cells as opposed to iPS cells.

The term “isolated” refers to a substance that is (i) separated from at least some other substances with which it is normally found in nature, usually by a process involving the hand of man, (ii) artificially produced (e.g., chemically synthesized), and/or (iii) present in an artificial environment or context (i.e., an environment or context in which it is not normally found in nature).

The term “nucleic acid” is used interchangeably with “polynucleotide” and encompasses in various embodiments naturally occurring polymers of nucleosides, such as DNA and RNA, and non-naturally occurring polymers of nucleosides or nucleoside analogs. In some embodiments a nucleic acid comprises standard nucleosides (abbreviated A, G, C, T, U). In other embodiments, a nucleic acid comprises one or more non-standard nucleosides. In some embodiments, one or more nucleosides are non-naturally occurring nucleosides or nucleotide analogs. A nucleic acid can comprise modified bases (for example, methylated bases), modified sugars (2′-fluororibose, arabinose, or hexose), modified phosphate groups or other linkages between nucleosides or nucleoside analogs (for example, phosphorothioates or 5′-N-phosphoramidite linkages), locked nucleic acids, or morpholinos. In some embodiments, a nucleic acid comprises nucleosides that are linked by phosphodiester bonds, as in DNA and RNA. In some embodiments, at least some nucleosides are linked by non-phosphodiester bond(s). A nucleic acid can be single-stranded, double-stranded, or partially double-stranded. An at least partially double-stranded nucleic acid can have one or more overhangs, e.g., 5′ and/or 3′ overhang(s). Nucleic acid modifications (e.g., nucleoside and/or backbone modifications, including use of non-standard nucleosides) known in the art as being useful in the context of RNA interference (RNAi), aptamer, or antisense-based molecules for research or therapeutic purposes are contemplated for use in various embodiments of the instant invention. See, e.g., Crooke, S T (ed.) Antisense drug technology: principles, strategies, and applications, Boca Raton: CRC Press, 2008; Kurreck, J. (ed.) Therapeutic oligonucleotides, RSC biomolecular sciences. Cambridge: Royal Society of Chemistry, 2008. In some embodiments, a modification increases half-life and/or stability of a nucleic acid, e.g., in vivo, relative to RNA or DNA of the same length and strandedness. In some embodiments, a modification decreases immunogenicity of a nucleic acid relative to RNA or DNA of the same length and strandedness. In some embodiments, between 5% and 95% of the nucleosides in one or both strands of a nucleic acid is modified. Modifications may be located uniformly or nonuniformly, and the location of the modifications (e.g., near the middle, near or at the ends, alternating, etc.) can be selected to enhance desired property(ies). A nucleic acid may comprise a detectable label, e.g., a fluorescent dye, radioactive atom, etc. “Oligonucleotide” refers to a relatively short nucleic acid, e.g., typically between about 4 and about 60 nucleotides long. Where reference is made herein to a polynucleotide, it is understood that both DNA, RNA, and in each case both single- and double-stranded forms (and complements of each single-stranded molecule) are provided. “Polynucleotide sequence” as used herein can refer to the polynucleotide material itself and/or to the sequence information (i.e. the succession of letters used as abbreviations for bases) that biochemically characterizes a specific nucleic acid. A polynucleotide sequence presented herein is presented in a 5′ to 3′ direction unless otherwise indicated.

The term “oligoclonal” refers to a population of cells that originated from a small population of cells, typically 2-1000 cells, that appear to share similar characteristics such as morphology or the presence or absence of markers of differentiation that differ from those of other cells in the same culture. Oligoclonal cells are isolated from cells that do not share these common characteristics, and are allowed to proliferate, generating a population of cells that are essentially entirely derived from the original population of similar cells.

The term “pluripotent stem cells” refers to animal cells capable of differentiating into more than one differentiated cell type. Such cells include hES cells, blastomere/morula cells and their derived hED cells, hiPS cells, hEG cells, hEC cells, and adult-derived cells including mesenchymal stem cells, neuronal stem cells, and bone marrow-derived stem cells. Pluripotent stem cells may be genetically modified or not genetically modified. Pluripotent stem cells may be in a naïve or primed state. Genetically modified cells may include markers such as fluorescent proteins or other markers to facilitate their identification or expression of particular genes of interest.

The term “polypeptide” refers to a polymer of amino acids. The terms “protein” and “polypeptide” are used interchangeably herein. A peptide is a relatively short polypeptide, typically between about 2 and 60 amino acids in length. Polypeptides used herein typically contain the standard amino acids (i.e., the 20 L-amino acids that are most commonly found in proteins). However, a polypeptide can contain one or more non-standard amino acids (which may be naturally occurring or non-naturally occurring) and/or amino acid analogs known in the art in certain embodiments. One or more of the amino acids in a polypeptide may be modified, for example, by the addition of a chemical entity such as a carbohydrate group, a phosphate group, a fatty acid group, a linker for conjugation, functionalization, etc. A polypeptide that has a nonpolypeptide moiety covalently or noncovalently associated therewith is still considered a “polypeptide”. Polypeptides may be purified from natural sources, produced using recombinant DNA technology, synthesized through chemical means such as conventional solid phase peptide synthesis, etc. The term “polypeptide sequence” or “amino acid sequence” as used herein can refer to the polypeptide material itself and/or to the sequence information (i.e., the succession of letters or three letter codes used as abbreviations for amino acid names) that biochemically characterizes a polypeptide. A polypeptide sequence presented herein is presented in an N-terminal to C-terminal direction unless otherwise indicated. A polypeptide may be cyclic or contain a cyclic portion. Where a naturally occurring polypeptide is discussed herein, it will be understood that the invention encompasses embodiments that relate to any isoform thereof (e.g., different proteins arising from the same gene as a result of alternative splicing or editing of mRNA or as a result of different alleles of a gene, e.g., alleles differing by one or more single nucleotide polymorphisms (typically such alleles will be at least 95%, 96%, 97%, 98%, 99%, or more identical to a reference or consensus sequence). A polypeptide may comprise a sequence that targets it for secretion or to a particular intracellular compartment (e.g., the nucleus) and/or a sequence targets the polypeptide for post-translational modification or degradation. Certain polypeptides may be synthesized as a precursor that undergoes post-translational cleavage or other processing to become a mature polypeptide. In some instances, such cleavage may only occur upon particular activating events. Where relevant, the invention provides embodiments relating to precursor polypeptides and embodiments relating to mature versions of a polypeptide.

The term “pooled clonal” refers to a population of cells obtained by combining two or more clonal populations to generate a population of cells with a uniformity of markers such as markers of gene expression, similar to a clonal population, but not a population wherein all the cells were derived from the same original clone. Said pooled clonal lines may include cells of a single or mixed genotypes. Pooled clonal lines are especially useful in the cases where clonal lines differentiate relatively early or alter in an undesirable way early in their proliferative lifespan.

The term “prenatal” refers to a stage of embryonic development of a placental mammal prior to which an animal is not capable of viability apart from the uterus.

The term “primordial stem cells” refers collectively to pluripotent stem cells capable of differentiating into cells of all three primary germ layers: endoderm, mesoderm, and ectoderm, as well as neural crest. Therefore, examples of primordial stem cells would include but not be limited by human or non-human mammalian ES cells or cell lines, blastomere/morula cells and their derived ED cells, iPS, and EG cells.

The term “purified” refers to agents or entities (e.g., compounds) that have been separated from most of the components with which they are associated in nature or when originally generated. In general, such purification involves action of the hand of man. Purified agents or entities may be partially purified, substantially purified, or pure. Such agents or entities may be, for example, at least 50%, 60%, 70%, 75%, 80%, 85%, 90%, 95%, 96%, 97%, 98%, 99%, or more than 99% pure. In some embodiments, a nucleic acid or polypeptide is purified such that it constitutes at least 75%, 80%, 855%, 90%, 95%, 96%, 97%, 98%, 99%, or more, of the total nucleic acid or polypeptide material, respectively, present in a preparation. Purity can be based on, e.g., dry weight, size of peaks on a chromatography tracing, molecular abundance, intensity of bands on a gel, or intensity of any signal that correlates with molecular abundance, or any art-accepted quantification method. In some embodiments, water, buffers, ions, and/or small molecules (e.g., precursors such as nucleotides or amino acids), can optionally be present in a purified preparation. A purified molecule may be prepared by separating it from other substances (e.g., other cellular materials), or by producing it in such a manner to achieve a desired degree of purity. In some embodiments, a purified molecule or composition refers to a molecule or composition that is prepared using any art-accepted method of purification. In some embodiments “partially purified” means that a molecule produced by a cell is no longer present within the cell, e.g., the cell has been lysed and, optionally, at least some of the cellular material (e.g., cell wall, cell membrane(s), cell organelle(s)) has been removed.

The term “RNA interference” (RNAi) is used herein consistently with its meaning in the art to refer to a phenomenon whereby double-stranded RNA (dsRNA) triggers the sequence-specific degradation or translational repression of a corresponding mRNA having complementarity to a strand of the dsRNA. It will be appreciated that the complementarity between the strand of the dsRNA and the mRNA need not be 100% but need only be sufficient to mediate inhibition of gene expression (also referred to as “silencing” or “knockdown”). For example, the degree of complementarity is such that the strand can either (i) guide cleavage of the mRNA in the RNA-induced silencing complex (RISC); or (ii) cause translational repression of the mRNA. In certain embodiments the double-stranded portion of the RNA is less than about 30 nucleotides in length, e.g., between 17 and 29 nucleotides in length. In certain embodiments a first strand of the dsRNA is at least 80%, 85%, 90%, 95%, or 100% complementary to a target mRNA and the other strand of the dsRNA is at least 80%, 85%, 90%, 95%, or 100% complementary to the first strand. In mammalian cells, RNAi may be achieved by introducing an appropriate double-stranded nucleic acid into the cells or expressing a nucleic acid in cells that is then processed intracellularly to yield dsRNA therein. Nucleic acids capable of mediating RNAi are referred to herein as “RNAi agents”. Exemplary nucleic acids capable of mediating RNAi are a short hairpin RNA (shRNA), a short interfering RNA (siRNA), and a microRNA precursor. These terms are well known and are used herein consistently with their meaning in the art. siRNAs typically comprise two separate nucleic acid strands that are hybridized to each other to form a duplex. They can be synthesized in vitro, e.g., using standard nucleic acid synthesis techniques. siRNAs are typically double-stranded oligonucleotides having 16-30, e.g., 16, 17, 18, 19, 20, 21, 22, 23, 24, 25, 26, 27, 28, 29, or 30 nucleotides (nt) in each strand, wherein the double-stranded oligonucleotide comprises a double-stranded portion between 15 and 29 nucleotides long and either or both of the strands may comprise a 3′ overhang between, e.g., 1-5 nucleotides long, or either or both ends can be blunt. In some embodiments, an siRNA comprises strands between 19 and 25 nt, e.g., between 21 and 23 nucleotides long, wherein one or both strands comprises a 3′ overhang of 1-2 nucleotides. One strand of the double-stranded portion of the siRNA (termed the “guide strand” or “antisense strand”) is substantially complementary (e.g., at least 80% or more, e.g., 85%, 90%, 95%, or 100%) complementary to (e.g., having 3, 2, 1, or 0 mismatched nucleotide(s)) a target region in the mRNA, and the other double-stranded portion is substantially complementary to the first double-stranded portion. In many embodiments, the guide strand is 100% complementary to a target region in an mRNA and the other passenger strand is 100% complementary to the first double-stranded portion (it is understood that, in various embodiments, the 3′ overhang portion of the guide strand, if present, may or may not be complementary to the mRNA when the guide strand is hybridized to the mRNA). In some embodiments, a shRNA molecule is a nucleic acid molecule comprising a stem-loop, wherein the double-stranded stem is 16-30 nucleotides long and the loop is about 1-10 nucleotides long. siRNA can comprise a wide variety of modified nucleosides, nucleoside analogs and can comprise chemically or biologically modified bases, modified backbones, etc. Without limitation, any modification recognized in the art as being useful for RNAi can be used. Some modifications result in increased stability, cell uptake, potency, etc. Some modifications result in decreased immunogenicity or clearance. In certain embodiments the siRNA comprises a duplex about 19-23 (e.g., 19, 20, 21, 22, or 23) nucleotides in length and, optionally, one or two 3′ overhangs of 1-5 nucleotides in length, which may be composed of deoxyribonucleotides. shRNA comprise a single nucleic acid strand that contains two complementary portions separated by a predominantly non-selfcomplementary region. The complementary portions hybridize to form a duplex structure and the non-selfcomplementary region forms a loop connecting the 3′ end of one strand of the duplex and the 5′ end of the other strand. shRNAs undergo intracellular processing to generate siRNAs. Typically, the loop is between 1 and 8, e.g., 2-6 nucleotides long.

MicroRNAs (miRNAs) are small, naturally occurring, non-coding, single-stranded RNAs of about 21-25 nucleotides (in mammalian systems) that inhibit gene expression in a sequence-specific manner. They are generated intracellularly from precursors (pre-miRNA) having a characteristic secondary structure comprised of a short hairpin (about 70 nucleotides in length) containing a duplex that often includes one or more regions of imperfect complementarity which is in turn generated from a larger precursor (pri-miRNA). Naturally occurring miRNAs are typically only partially complementary to their target mRNA and often act via translational repression. RNAi agents modelled on endogenous miRNA or miRNA precursors are of use in certain embodiments of the invention. For example, an siRNA can be designed so that one strand hybridizes to a target mRNA with one or more mismatches or bulges mimicking the duplex formed by a miRNA and its target mRNA. Such siRNA may be referred to as miRNA mimics or miRNA-like molecules. miRNA mimics may be encoded by precursor nucleic acids whose structure mimics that of naturally occurring miRNA precursors.

In certain embodiments an RNAi agent is a vector (e.g., a plasmid or virus) that comprises a template for transcription of an siRNA (e.g., as two separate strands that can hybridize to each other), shRNA, or microRNA precursor. Typically the template encoding the siRNA, shRNA, or miRNA precursor is operably linked to expression control sequences (e.g., a promoter), as known in the art. Such vectors can be used to introduce the template into vertebrate cells, e.g., mammalian cells, and result in transient or stable expression of the siRNA, shRNA, or miRNA precursor. Precurors (shRNA or miRNA precursors) are processed intracellularly to generate siRNA or miRNA.

In general, small RNAi agents such as siRNA can be chemically synthesized or can be transcribed in vitro or in vivo from a DNA template either as two separate strands that then hybridize, or as an shRNA which is then processed to generate an siRNA. Often RNAi agents, especially those comprising modifications, are chemically synthesized. Chemical synthesis methods for oligonucleotides are well known in the art.

The term “small molecule” as used herein, is an organic molecule that is less than about 2 kilodaltons (KDa) in mass. In some embodiments, the small molecule is less than about 1.5 KDa, or less than about 1 KDa. In some embodiments, the small molecule is less than about 800 daltons (Da), 600 Da, 500 Da, 400 Da, 300 Da, 200 Da, or 100 Da. Often, a small molecule has a mass of at least 50 Da. In some embodiments, a small molecule contains multiple carbon-carbon bonds and can comprise one or more heteroatoms and/or one or more functional groups important for structural interaction with proteins (e.g., hydrogen bonding), e.g., an amine, carbonyl, hydroxyl, or carboxyl group, and in some embodiments at least two functional groups. Small molecules often comprise one or more cyclic carbon or heterocyclic structures and/or aromatic or polyaromatic structures, optionally substituted with one or more of the above functional groups. In some embodiments, a small molecule is non-polymeric. In some embodiments, a small molecule is not an amino acid. In some embodiments, a small molecule is not a nucleotide. In some embodiments, a small molecule is not a saccharide.

The term “subject” can be any multicellular animal. Often a subject is a vertebrate, e.g., a mammal or avian. Exemplary mammals include, e.g., humans, non-human primates, rodents (e.g., mouse, rat, rabbit), ungulates (e.g., ovine, bovine, equine, caprine species), canines, and felines. Often, a subject is an individual to whom a compound is to be delivered, e.g., for experimental, diagnostic, and/or therapeutic purposes or from whom a sample is obtained or on whom a diagnostic procedure is performed (e.g., a sample or procedure that will be used to assess tissue damage and/or to assess the effect of a compound of the invention).

The term “treat”, “treating”, “therapy”, “therapeutic” and similar terms in regard to a subject refer to providing medical and/or surgical management of the subject. Treatment can include, but is not limited to, administering a compound or composition (e.g., a pharmaceutical composition) to a subject. Treatment of a subject according to the instant invention is typically undertaken in an effort to promote regeneration, e.g., in a subject who has suffered tissue damage or is expected to suffer tissue damage (e.g., a subject who will undergo surgery). The effect of treatment can generally include increased regeneration, reduced scarring, and/or improved structural or functional outcome following tissue damage (as compared with the outcome in the absence of treatment), and/or can include reversal or reduction in severity or progression of a degenerative disease.

The term “variant” as applied to a particular polypeptide refers to a polypeptide that differs from such polypeptide (sometimes referred to as the “original polypeptide”) by one or more amino acid alterations, e.g., addition(s), deletion(s), and/or substitution(s). Sometimes an original polypeptide is a naturally occurring polypeptide (e.g., from human or non-human animal) or a polypeptide identical thereto. Variants may be naturally occurring or created using, e.g., recombinant DNA techniques or chemical synthesis. An addition can be an insertion within the polypeptide or an addition at the N- or C-terminus. In some embodiments, the number of amino acids substituted, deleted, or added can be for example, about 1 to 30, e.g., about 1 to 20, e.g., about 1 to 10, e.g., about 1 to 5, e.g., 1, 2, 3, 4, or 5. In some embodiments, a variant comprises a polypeptide whose sequence is homologous to the sequence of the original polypeptide over at least 50 amino acids, at least 100 amino acids, at least 150 amino acids, or more, up to the full length of the original polypeptide (but is not identical in sequence to the original polypeptide), e.g., the sequence of the variant polypeptide is at least 50%, 60%, 70%, 75%, 80%, 85%, 90%, 95%, 96%, 97%, 98%, 99%, or more identical to the sequence of the original polypeptide over at least 50 amino acids, at least 100 amino acids, at least 150 amino acids, or more, up to the full length of the original polypeptide. In some embodiments, a variant comprises a polypeptide at least 50%, 60%, 70%, 75%, 80%, 85%, 90%, 91%, 92%, 93%, 94%, 95%, 96%, 97%, 98%, 99%, 99.5% or more identical to an original polypeptide over at least 50%, 60%, 70%, 80%, 85%, 90%, 91%, 92%, 93%, 94%, 95%, 96%, 97%, 98%, 99%, or 100% of the length of the original polypeptide. In some embodiments, a variant comprises at least one functional or structural domain, e.g., a domain identified as such in the Conserved Domain Database (CDD) of the National Center for Biotechnology Information (www.ncbi.nih.gov), e.g., an NCBI-curated domain.

In some embodiments one, more than one, or all biological functions or activities of a variant or fragment is substantially similar to that of the corresponding biological function or activity of the original molecule. In some embodiments, a functional variant retains at least 10%, 20%, 30%, 40%, 50%, 60%, 70%, 80%, 90%, 95%, 96%, 97%, 98%, 99%, or more of the activity of the original polypeptide, e.g., about equal activity. In some embodiments, the activity of a variant is up to approximately 100%, approximately 125%, or approximately 150% of the activity of the original molecule. In other nonlimiting embodiments, an activity of a variant or fragment is considered substantially similar to the activity of the original molecule if the amount or concentration of the variant needed to produce a particular effect is within 0.5 to 5-fold of the amount or concentration of the original molecule needed to produce that effect.

In some embodiments amino acid “substitutions” in a variant are the result of replacing one amino acid with another amino acid having similar structural and/or chemical properties, i.e., conservative amino acid replacements. “Conservative” amino acid substitutions may be made on the basis of similarity in any of a variety or properties such as side chain size, polarity, charge, solubility, hydrophobicity, hydrophilicity, and/or amphipathicity of the residues involved. For example, the non-polar (hydrophobic) amino acids include alanine, leucine, isoleucine, valine, glycine, proline, phenylalanine, tryptophan and methionine. The polar (hydrophilic), neutral amino acids include serine, threonine, cysteine, tyrosine, asparagine, and glutamine. The positively charged (basic) amino acids include arginine, lysine and histidine. The negatively charged (acidic) amino acids include aspartic acid and glutamic acid. Within a particular group, certain substitutions may be of particular interest, e.g., replacements of leucine by isoleucine (or vice versa), serine by threonine (or vice versa), or alanine by glycine (or vice versa). Of course, non-conservative substitutions are often compatible with retaining function as well. In some embodiments, a substitution or deletion does not alter or delete an amino acid important for activity. Insertions or deletions may range in size from about 1 to 20 amino acids, e.g., 1 to 10 amino acids. In some instances, larger domains may be removed without substantially affecting function. In certain embodiments of the invention the sequence of a variant can be obtained by making no more than a total of 5, 10, 15, or 20 amino acid additions, deletions, or substitutions to the sequence of a naturally occurring enzyme. In some embodiments no more than 1%, 5%, 10%, or 20% of the amino acids in a polypeptide are insertions, deletions, or substitutions relative to the original polypeptide. Guidance in determining which amino acid residues may be replaced, added, or deleted without eliminating or substantially reducing activities of interest, may be obtained by comparing the sequence of the particular polypeptide with that of homologous polypeptides (e.g., from other organisms) and minimizing the number of amino acid sequence changes made in regions of high homology (conserved regions) or by replacing amino acids with those found in homologous sequences since amino acid residues that are conserved among various species are more likely to be important for activity than amino acids that are not conserved.

In some embodiments, a variant of a polypeptide comprises a heterologous polypeptide portion. The heterologous portion often has a sequence that is not present in or homologous to the original polypeptide. A heterologous portion may be, e.g., between 5 and about 5,000 amino acids long, or longer. Often it is between 5 and about 1,000 amino acids long. In some embodiments, a heterologous portion comprises a sequence that is found in a different polypeptide, e.g., a functional domain. In some embodiments, a heterologous portion comprises a sequence useful for purifying, expressing, solubilizing, and/or detecting the polypeptide. In some embodiments, a heterologous portion comprises a polypeptide “tag”, e.g., an affinity tag or epitope tag. For example, the tag can be an affinity tag (e.g., HA, TAP, Myc, 6×His, Flag, GST), fluorescent or luminescent protein (e.g., EGFP, ECFP, EYFP, Cerulean, DsRed, mCherry), solubility-enhancing tag (e.g., a SUMO tag, NUS A tag, SNUT tag, or a monomeric mutant of the Ocr protein of bacteriophage T7). See, e.g., Esposito D and Chatterjee D K. Curr Opin Biotechnol.; 17(4):353-8 (2006). In some embodiments, a tag can serve multiple functions. A tag is often relatively small, e.g., ranging from a few amino acids up to about 100 amino acids long. In some embodiments a tag is more than 100 amino acids long, e.g., up to about 500 amino acids long, or more. In some embodiments, a polypeptide has a tag located at the N- or C-terminus, e.g., as an N- or C-terminal fusion. The polypeptide could comprise multiple tags. In some embodiments, a 6.times.His tag and a NUS tag are present, e.g., at the N-terminus. In some embodiments, a tag is cleavable, so that it can be removed from the polypeptide, e.g., by a protease. In some embodiments, this is achieved by including a sequence encoding a protease cleavage site between the sequence encoding the portion homologous to the original polypeptide and the tag. Exemplary proteases include, e.g., thrombin, TEV protease, Factor Xa, PreScission protease, etc. In some embodiments, a “self-cleaving” tag is used. See, e.g., PCT/US05/05763. Sequences encoding a tag can be located 5′ or 3′ with respect to a polynucleotide encoding the polypeptide (or both). In some embodiments, a tag or other heterologous sequence is separated from the rest of the polypeptide by a polypeptide linker. For example, a linker can be a short polypeptide (e.g., 15-25 amino acids). Often a linker is composed of small amino acid residues such as serine, glycine, and/or alanine. A heterologous domain could comprise a transmembrane domain, a secretion signal domain, etc.

In certain embodiments of the invention a fragment or variant, optionally excluding a heterologous portion, if present, possesses sufficient structural similarity to the original polypeptide so that when its 3-dimensional structure (either actual or predicted structure) is superimposed on the structure of the original polypeptide, the volume of overlap is at least 70%, preferably at least 80%, more preferably at least 90% of the total volume of the structure of the original polypeptide. A partial or complete 3-dimensional structure of the fragment or variant may be determined by crystallizing the protein, which can be done using standard methods. Alternately, an NMR solution structure can be generated, also using standard methods. A modeling program such as MODELER (Sali, A. and Blundell, T L, J. Mol. Biol., 234, 779-815, 1993), or any other modeling program, can be used to generate a predicted structure. If a structure or predicted structure of a related polypeptide is available, the model can be based on that structure. The PROSPECT-PSPP suite of programs can be used (Guo, J T, et al., Nucleic Acids Res. 32 (Web Server issue):W522-5, Jul. 1, 2004). Where embodiments of the invention relate to variants of a polypeptide, it will be understood that polynucleotides encoding the variant are provided.

The term “vector” is used herein to refer to a nucleic acid or a virus or portion thereof (e.g., a viral capsid or genome) capable of mediating entry of, e.g., transferring, transporting, etc., a nucleic acid molecule into a cell. Where the vector is a nucleic acid, the nucleic acid molecule to be transferred is generally linked to, e.g., inserted into, the vector nucleic acid molecule. A nucleic acid vector may include sequences that direct autonomous replication (e.g., an origin of replication), or may include sequences sufficient to allow integration of part or all of the nucleic acid into host cell DNA. Useful nucleic acid vectors include, for example, DNA or RNA plasmids, cosmids, and naturally occurring or modified viral genomes or portions thereof or nucleic acids (DNA or RNA) that can be packaged into viral) capsids. Plasmid vectors typically include an origin of replication and one or more selectable markers. Plasmids may include part or all of a viral genome (e.g., a viral promoter, enhancer, processing or packaging signals, etc.). Viruses or portions thereof that can be used to introduce nucleic acid molecules into cells are referred to as viral vectors. Useful viral vectors include adenoviruses, adeno-associated viruses, retroviruses, lentiviruses, vaccinia virus and other poxviruses, herpesviruses (e.g., herpes simplex virus), and others. Viral vectors may or may not contain sufficient viral genetic information for production of infectious virus when introduced into host cells, i.e., viral vectors may be replication-defective, and such replication-defective viral vectors may be preferable for therapeutic use. Where sufficient information is lacking it may, but need not be, supplied by a host cell or by another vector introduced into the cell. The nucleic acid to be transferred may be incorporated into a naturally occurring or modified viral genome or a portion thereof or may be present within the virus or viral capsid as a separate nucleic acid molecule. It will be appreciated that certain plasmid vectors that include part or all of a viral genome, typically including viral genetic information sufficient to direct transcription of a nucleic acid that can be packaged into a viral capsid and/or sufficient to give rise to a nucleic acid that can be integrated into the host cell genome and/or to give rise to infectious virus, are also sometimes referred to in the art as viral vectors. Vectors may contain one or more nucleic acids encoding a marker suitable for use in the identifying and/or selecting cells that have or have not been transformed or transfected with the vector. Markers include, for example, proteins that increase or decrease either resistance or sensitivity to antibiotics (e.g., an antibiotic-resistance gene encoding a protein that confers resistance to an antibiotic such as puromycin, hygromycin or blasticidin) or other compounds, enzymes whose activities are detectable by assays known in the art (e.g., beta.-galactosidase or alkaline phosphatase), and proteins or RNAs that detectably affect the phenotype of transformed or transfected cells (e.g., fluorescent proteins). Expression vectors are vectors that include regulatory sequence(s), e.g., expression control sequences such as a promoter, sufficient to direct transcription of an operably linked nucleic acid. Regulatory sequences may also include enhancer sequences or upstream activator sequences. Vectors may optionally include 5′ leader or signal sequences. Vectors may optionally include cleavage and/or polyadenylations signals and/or a 3′ untranslated regions. Vectors often include one or more appropriately positioned sites for restriction enzymes, to facilitate introduction into the vector of the nucleic acid to be expressed. An expression vector comprises sufficient cis-acting elements for expression; other elements required or helpful for expression can be supplied by the host cell or in vitro expression system.

Various techniques may be employed for introducing nucleic acid molecules into cells. Such techniques include chemical-facilitated transfection using compounds such as calcium phosphate, cationic lipids, cationic polymers, liposome-mediated transfection, non-chemical methods such as electroporation, particle bombardment, or microinjection, and infection with a virus that contains the nucleic acid molecule of interest (sometimes termed “transduction”). Markers can be used for the identification and/or selection of cells that have taken up the vector and, typically, express the nucleic acid. Cells can be cultured in appropriate media to select such cells and, optionally, establish a stable cell line.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order that is logically possible.

The present invention provides an improved in vitro model for detection of human cardiac drug responses using human pluripotent stem cell-derived cardiomyocytes (hPSC-CM). The developmental immaturity of hPSC-CM is a primary limitation of prior models for cardiotoxicity screening of drugs. In the present invention, hPSC-CM with improved maturity is provided with increased utility.

hPSC-CM display the typical morphological and genetic expression characteristics of fetal cardiomyocytes (see Table 1 below) leading to host of inappropriate electrical-mechanical behavior.

TABLE 1

| hPSC Derived Cardiomyocytes | Mature Cardiomyocytes |
|---|---|
| Morphology round (aspect ratio 2-3:1) or | Morphology rod shaped (aspect ratio of 5-9.5:1), |
| polygonal, chaotically organized and very | longitudinally aligned and ~30% cells bi or poly- |
| limited bi-nucleation. | nuclear. |
| Sarcomeres disorganized with mainly Z-discs | Sarcomeres organized with Z-discs, I, H, A and |
| and I-bands. Sarcomere length 1.6 microns. | M bands. Sarcomere length 2.2 microns. |
| Expression of sarcomere proteins less than | Express sarcomere proteins CSQ, PLN, RYR2, |
| adult. | SERCA/ATP2A2 |
| Gene expression profile like first-trimester | Gene expression patterns are “mature” for |
| gestational stage cardiomyocytes. | ion-channels and contractile protein encoding. |
| MYH6 (α-MHC) > MYH7 (β-MHC) (h | MYH7 (β-MHC) > MYH6 (α-MHC) (h ventric. |
| ventric. CM) | CM) |
| TNNI1 (fetal ssTnI troponin) > TNNI3 (cTnI). | TNNI3 (cTnI) > TNNI1 (fetal ssTnI troponin) |
| ADRA1A (α-adrenoceptor) not expressed. | ADRA1A (α-adrenoceptor) not expressed. |
| Predominately use glycolysis via anaerobic | Preferentially generate energy via fatty-acid |
| glycolysis for energy production. | oxidation to produce acetyl-CoA (via beta |
| | oxidation). |
| Mitochondria throughout cell; occupies 20- | Mitochondria near nuclei |
| 40% of cell volume. | |
| Resting membrane potential −50 to −70 mV. | Resting membrane potential −90 mV (ventricular). |
| Relatively depolarized due to low or absent | |
| inward rectifier K+ current. | |
| Reduced fractional shortening. Force of 40 to | Fractional shortening normal. Force of 0.08 to 4 |
| 80 mN/mm2. | mN/mm2. |
| Lack transverse (t) tubules. | Mature t-tubules present. |
| Exhibit “automaticity” or spontaneous | Do not exhibit automaticity. |
| beating. | |
| Single nucleus | Multi-nucleated |

The degree of maturity of hPSC-CM limits the usefulness of these models. The sensitivity and accuracy of the use of hPSC-CM models for drug toxicity testing would be improved by generating cardiomyocytes that more closely resemble those in adults. For example, preventing h-PSC-CM from using glucose is particularly important for cardiotoxicity analysis, as drugs that impair mitochondrial function will not be well detected in glycolysis-dependent cells. In addition, for testing drugs for toxicity involving contractile function or structural remodeling, the degree to which cardiotoxicity signaling pathways in hPSC-derived cardiomyocytes recapitulate those in adult cardiomyocytes is critical, and the immaturity of current models renders deficient them deficient in these areas (Magdy et al).

Adult cardiomyocytes obtain most of their energy production by oxidative metabolism, while hPSC-CM use mostly glycolysis (a characteristic of immature cardiomyocytes). Indeed, it has been shown that establishment of the mitochondrial system and engagement of oxidative metabolism are prerequisites for the differentiation of stem cells into a functional cardiac phenotype (Chung). Preventing hPSC-CM from using glucose is particularly important for cardiotoxicity analysis, as drugs that impair mitochondrial functions will not be well detected in glycolysis-dependent cells (Magdy). The in vitro assay featuring metabolically mature hPSC-CMs according to the invention improves early detection rates of cardiotoxic effects.

hPSC-CM can be brought to an improved level of metabolic, structural and functional maturity by modifying the hPSC-CM cellular energy production to reduce the use of anaerobic glycolysis to greater reliance on fatty acid oxidation to produce acetyl-CoA through the constitutive expression of COX7A1.

COX7A1 encodes a heart and muscle-specific isoform of the subunit VIIA of cytochrome c oxidase (COX), the terminal complex of the mitochondrial electron transport chain. Its relatively high expression in cardiac and skeletal muscle suggests a role in increased oxidative phosphorylation and ATP production, though knockout of the gene is reported to not increase ATP levels. Nevertheless, COX7A1 knockout mice develop a mild form of dilated cardiomyopathy that resolves over time (Huttemann). The expression of the gene COX7A1 is associated with the embryonal-to-fetal transition (EFT)(West). The EFT occurs at eight weeks of human development and is marked by the commitment of cells to a more differentiated phenotype.

Applying deep neural network (DNN) ensembles, the accuracy of DNN ensembles in classifying embryonic vs. adult cells has been demonstrated. Novel markers associated with the EFT were assembled and it was observed that COX7A1 expression was highly associated with the fetal/adult state as compared to the embryonic state.

We determine that glycolytic intermediates are lower in embryonic stem cell-derived clonal embryonic progenitor cell lines such as 4D20.8 and cancer cells such as HT1080 following the exogenous expression of COX7A1. Glycolytic metabolites declined up to 90% following COX7A1 expression in cells, regardless of cell type (Table 2).

TABLE 2
Glycolytic metabolism showing the ratio of expressing
cells compared to cells expressing only eGFP: SI =
Significantly Increased (p ≤ 0.05); TI = Trending
Increased (0.05 < p < 0.1); SD = Significantly
Decreased (p ≤ 0.05); and TD—Trending Decreased (0.05 < p < 0.1).

| | 4D20.8_COX7A1 | HT1080_COX7A1 |
|---|---|---|
| Biochemical Name | 4D20.8_eGFP | HT1080_eGFP |
| glucose | 1.29 | 1.01 |
| glucose 6-phosphate | 0.53 | 0.35 |
| | SD | SD |
| fructose 1,6-diphosphate/ | 0.01 | 0.68 |
| glucose 1,6-diphosphate | SD | |
| dihydroxyacetone phosphate | 0.19 | 0.62 |
| (DHAP) | SD | SD |
| 3-phosphoglycerate | 0.22 | 0.32 |
| | TD | SD |
| phosphoenolpyruvate (PEP) | 0.2 | 0.65 |
| | SD | |
| pyruvate | 1.92 | 1.4 |
| | SI | TI |
| lactate | 1.18 | 0.96 |
| glycerate | 3.04 | 0.71 |
| | SI | |

Diminished glycolytic metabolites decreased glucose uptake and/or glucose metabolism; however, entry into the TCA cycle could also be impaired as evidenced by increased cellular pyruvate. Together, these data illustrate that cellular energy production from glucose is diminished when COX7A1 is constitutively expressed in embryonic and cancer cells.

Cells constitutively expressing COX7A1 were found to increase fat oxidation. COX7A1 knock-in resulted in elevations of long chain acylcarnitines, suggesting increased transport into the mitochondria (Table 3).

TABLE 3
Metabolic pathway and box plots of the fatty acid oxidation
showing ratio of values in COX7A1 expressing compared
to eGFP expressing cells: SI = Significantly Increased
(p ≤ 0.05); TI = Trending Increased (0.05 <
p < 0.1); SD = Significantly Decreased (p ≤
0.05); and TD—Trending Decreased (0.05 < p <
0.1) biochemicals respectively.

| | 4D20.8_COX7A1 | HT1080_COX7A1 |
|---|---|---|
| Biochemical Name | 4D20.8_eGFP | HT1080_eGFP |
| hexanoylcarnitine (C6) | 0.57 | 1.24 |
| | SD | |
| octanoylcarnitine (C8) | 0.48 | 0.92 |
| | SD | |
| laurylcarnitine (C12) | 1.25 | 1.29 |
| myristoylcarnitine (C14) | 1.09 | 1.53 |
| | | TI |
| pentadecanoylcarnitine (C15)* | 1.18 | 1.61 |
| palmitoylcarnitine (C16) | 1.28 | 1.64 |
| | TI | TI |
| margaroylcarnitine (C17)* | 1.8 | 1.54 |
| | SI | |
| stearoylcamitine (C18) | 2.69 | 1.44 |
| | SI | TI |
| arachidoylcarnitine (C20)* | 2.65 | 1.82 |
| | SI | TI |
| behenoylcarnitine (C22)* | 2.96 | 1 |
| | SI | |
| lignoceroylcarnitine (C24)* | 2.73 | 1 |
| | SI | |
| cerotoylcarnitine (C26)* | 0.8 | 0.56 |
| | TD | |

Consistent with elevated fatty acid oxidation, 3-hydroxybutyrate levels were 1.75 and 2.45-fold higher in COX7A1 expressing embryonic and cancer cells than in controls, although differences did not reach statistical significance in embryonic cells. Taken together, these data demonstrate that expression of COX7A1 switches cellular fuel preferences by increasing fatty acid oxidation and diminishing glucose oxidation.

The ESI-017 hESC line (ESI-017) has been approved by NIH to be used in government-funded research due to proper documentation and derivation protocols. Using known scalable protocols, ESI-017 has been shown to differentiate into CM in robust fashion and can be cryopreserved for future use as CM cultures. ESI-017 has been transfected with an inducible cassette placed in a safe harbor, that expresses COX7A1 (CX7-ESI-017).

In the method according to the invention, the expression of COX7A1 leads to hESC derived cells that decrease the use of glucose as an energy source and preferentially upregulate the use fatty-acid oxidation. The CM produced by this method demonstrate a more mature and physiologically relevant phenotype to be used in assays for cardiotoxicity testing. In a preferred embodiment, ESI-017 is differentiated to CM that can be induced to expresses COX7A1. In a further preferred embodiment that CM is CX7-ESI-017 CM is derived from CX7-ESI-017.

In a further preferred embodiment, an assay is provided that features more metabolically mature populations of CM as measured by their utilization of fatty acids as an energy source as opposed to existing hPSC-derived CM products that primarily utilize glucose as an energy source.

The invention is further directed to a cardiotoxicity assay and method of conducting a cardiotoxicity assay that uses a micropatterned well.

In one embodiment, the invention is directed to a single or multi-well cell culture vessel containing extra cellular matrix materials micropatterned onto the surface of one or more wells. The wells may contains at least two types of human pluripotent stem cell (hPSC) derived cardiomyocytes. In a further preferred embodiment, each well further contains at least two vascular cell types. The well may further contains a culture medium.

The hPSC may be one of the following: human embryonic stem cells; human induced pluripotent stem cells (hiPSC); human parthenogenetic stem cells; a hPSC that has been transfected to express COX7A1 and/or the let-7 family of miRNAs.

The hPSC-derived cardiomyocytes can be nodal cells, ventricular cardiomyocytes, and/or atrial cardiomyocytes. The vascular cell types can be vascular endothelial cell; hPSC-derived pericyte; hPSC-derived vascular smooth muscle cell; and/or human embryonic vascular progenitor cell. In a preferred embodiment, the hPSC-derived pericyte is derived from ESI-017.

In another preferred embodiment, the single or multi-well plate contains a polydimethylsiloxane (PDMS) coated surface.

In another preferred embodiment, the micropatterned matrix is made up of different thicknesses of one or a combination of the following types of extracellular matrices: fibronectin, collagen type I, collagen type III, laminin, hyaluronan, proteoglycans, elastin, fibrillin, and tenascin. In a further preferred embodiment, the micropatterned matrix is made of fibronectin.

This cardiac extracellular matrix (ECM) provides an environment that provides for improved cardiac development, cardiomyocyte maturation and electro-mechanical function. The ECM provides a guiding structure allowing the cardiomyocytes to align themselves from “end to end” along their long axis, an orientation that enables the myocardium to act as a syncytium. The resulting tissue structure promotes the formation of intercalated discs between the cells for proper action potential (AP) propagation. Orienting the cells along their long axis thereby directing the force of cellular contraction parallel to the muscle fiber, promotes the quality of cardiac tissue called “anisotropy” (different physical properties along different axes). The ECM scaffold organizes the cells and accommodates contraction and relaxation and facilitates force transduction, electrical conductance, intercellular communication and metabolic exchange within the myocardial environment (Spreeuwel et al).

In a preferred embodiment, the ECM has an elastic modulus that ranges from 10 kPa to 90 kPa.

In another preferred embodiment, the ECM contains peptides for the specific attachment of human pluripotent stem cell derived vascular or atrial cardiomyocytes.

In another preferred embodiment, the ECM contains peptides for the specific attachment of human pluripotent stem cell derived nodal cells.

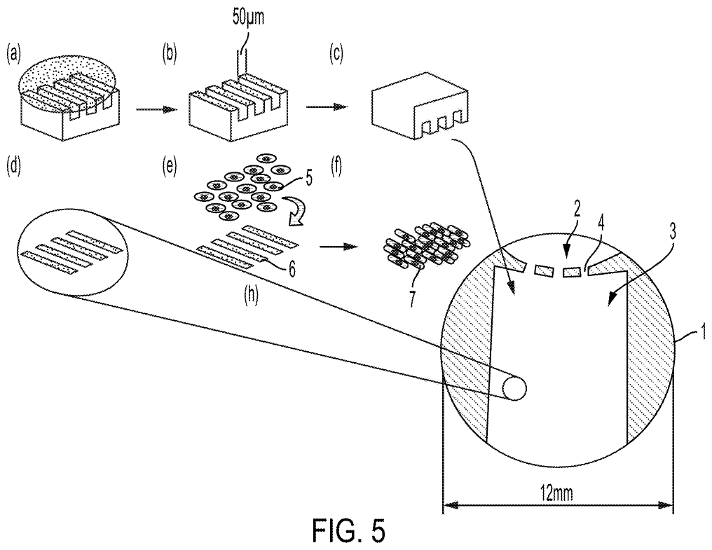

In one embodiment, the micropattern separates the hPSC derived nodal cells from the population of atrial or ventricular cardiomyocytes by a gated barrier of ECM. For example, as shown in FIG. 5, a well (1) is separated into two zones by a gated barrier (4). In a first zone (2), hPSC derived nodal cells are placed. In a second zone (3), vascular or atrial cardiomyocytes (5) are placed in elongated furrows (6), which preferably elongate the cells (7). The micropattern in the area of vascular or atrial cardiomyocytes (3) is composed of parallel lines of matrix material that are raised up and separated by trenches. For example, the lines can be between about 10 and about 60 microns in diameter, and the trench can be between about 10 and about 60 microns in diameter. The lines and spaces can further vary between about 10 and about 40 microns where the lines come together at the intersection with the nodal island. The gates in the gated barrier (4) between the zones (2, 3) have a diameter of about 1 to about 12 mm.

In another preferred embodiment, at least some of the human stem cell derived cardiomyocytes are in contact with each other and aligned in a parallel orientation forming a syncytium. In another preferred embodiment, at least some of the human stem cell derived cardiomyocytes are connected to each other through intercalated discs.

The human stem cell derived cardiomyocytes preferably exhibit active voltage-gated sodium (Na⁺) and calcium (Ca⁺⁺) channels and active potassium channels.

The human stem cell derived nodal cells preferably contact the human stem cell derived cardiomyocytes at at least one point, and further preferably make contact at multiple points.

In a further preferred embodiment, the human stem cell derived cardiomyocytes exhibit contraction rate that is modulated and synchronized by the human stem cell derived nodal cells, such rate in this composition is between 60 and 100 contractions per minute.

The medium preferably contains at least one of the following supplements: VEGF, bFGF, SDF-1, GM-CSF, tri-iodo-1-thyronine (T3), insulin, glucocorticoids (such as dexamethasone) and phosphodiesterase inhibitor 3-isobutyl-1-methil-xanthine.

The micropattern can be made according to the methods described in U.S. Pat. No. 8,449,285, which is incorporated herein in its entirety by reference.

In a preferred embodiment, the micropattern can be prepared by microcontact printing of the ECM protein fibronectin (FN) onto polydimethylsiloxane (PDMS) coated glass slides using 2D photolithographic masks. See FIG. 5. A PDMS multi-head stamp can be created with a pattern consisting of lines and spaces varying between 10 and 60 microns where the lines come together at the intersection with the nodal island. These stamps can be made using AutoCAD methods.

The PDMS stamp patterned surfaces incubated with 25 ug/mL drops of FN for one hour and dried with a nitrogen gun. The FN patterns can be transferred into the surface of a UV-ozone treated (UVO) PDMS hydrophilic surface at the bottom of a glass multi-well plate by bringing the surface of a dry multi-head stamp in contact with the surface. The FN patterns can be transferred into the surface of a UV-ozone treated (UVO) PDMS hydrophilic surface at the bottom of a glass multi-well plate by bringing the surface of the dry multi-head stamp in contact with the surface. A background application of 2.5 ug/mL FN is added directly onto the well surfaces providing lines of high density FN against spaces of lower density FN. Wells can be seeded, for example, with 1×10⁶ cells at a concentration of 5×10⁵ cells per mL.

Another aspect of the present invention is coculturing with secondary cell types to improve PSC-CM maturing and function. The likely mechanism of cell-cell communication leading to improvements in maturity and function is though messages carried from cell to cell in micro-vesicles (MVs) or through the excretion of various soluble factors. In normal adult hearts, fibroblasts are the most populous non-myocyte cell type and account for 20% of the myocardial volume (Dobaczewski). It is thought that the fibroblasts contribute to the preservation of the cardiac ECM and may directly interact with CMs as active participants in cardiac function, however there is little published evidence that co-culture with cardio-fibroblasts improves maturity of hPSC-CM. Other cell types present are vascular cells, including endothelial cells, smooth muscle cells and pericytes. CMs derived from mouse ESC (mESC-CM) express a more mature phenotype when co-cultured with rat aortic endothelial cells. The co-cultures display an elongated shape, mature aligned Z banding of myofibrils, a switch from the fetal-rodent-specific myosin heavy-chain beta isoform to the adult-rodent-specific alpha isoform and improved functionality of calcium clearance from the cytosol (Lee et al). The co-culture system was shown to upregulate the expression of four miRNAs (miRNA-125b, miR-199a, miR-221, miR-222). When these four miRNAs are overexpressed in transfected mono-cultures of mESC-CM expressing these miRNAs, the CMs exhibited the same mature phenotype as they did in the co-culture. Interestingly, the co-culture system promoted maturation of the mESC-CM regardless of the source of the endothelial cells (heart, fat or aorta) or the species (mouse, rat, pig or human). Human iPSC-CM co-cultured with human bone-marrow-derived mesenchymal stem cells (hMSC) show significant improvement in maturity as compared with hiPSC-CM in mono-culture (rod-shaped morphology with fully formed mitochondria and aligned myofibrils with A-, H- and I-bands and an increase in the MHC-β to MHC-α ratio) (Lee). The hMSC produce soluble factors (VEGF, bFGF, SDF-1 and GM-CSF) and microsomes containing miRNAs (let7 family, miR-134, miR-145, miR-296) that were individually found to contribute to the maturation of hiPSC-CMs (Lee et al). The overexpression of the let-7 family members of miRNAs enhanced cell size, sarcomere length, force of contraction and respiratory capacity in hESC-CM (Kuppusamy, et al.). Other published research has found “large quantities” of extracellular micro-vesicles in decellularized atrial tissue. These micro-vesicles contain miRNA miR-199-3p (An et al) and were found to promote cell growth of isolated neonatal CMs and sinus nodal cells and to enhance the electrocardiographic signal activity of sinus nodal cells (ibid). Mouse embryonic stem cells secrete exosomes shown to augment the survival, retention, and proliferation of cardiac progenitor cells in ischemic myocardium by delivery of miR-294 (Sluijter et al). A modified CM differentiation protocol that replaces the Wnt antagonist Dkk1 at day 5 with vascular endothelial growth factor (VEGF) from day 5-day 15 produces a mixed culture containing CMs and two types of vascular cells, CD144 positive vascular endothelial cells (EC) and CD140b positive “mural cells” (a type of vascular smooth muscle/pericyte). This mixed culture was approximately 62% CM, 19% EC and 2% MC and shows improved force measurement and improved sarcomere structure relative to CM cultures lacking vascular cells (Masumoto et al).

Another aspect of the invention is biophysical cues and soluble factors. Biophysical cues such as electrical stimulation of beating or matrix stiffness have been shown to improve hPSC-CM maturity in culture. hPSC-CM cultured in 3D cultures, when stimulated with repetitive electrical fields to simulate nodal pacemaker excitation showed elongated morphology, increased sarcomere volume, increased number of mitochondria and intercalated discs, gap junctions and contractility (Nunes, et al.). Studies looking at variations of stiffness of the ECM and hPSC-CM maturity show that increasing the stiffness enhances maturation, however there are great discrepancies in the elastic modulus levels that are optimal with ranges varying from 10 kPa to 90 kPa. It is possible these discrepancies reflect the difference approaches used to measure elastic modulus (Denning, et al.)

Several medium supplements have been shown to improve CM, including the soluble factors VEGF, bFGF, SDF-1 and GM-CSF produced by bone marrow MSC as mentioned earlier. Tri-iodo-1-thyronine (T3) is an essential thyroid hormone for normal heart development and has been shown to improve the maturity of hiPSC-CM in culture, showing improved force per-beat after T3 treatment and also an enhancement in contractile kinetics. This improvement in force generation was accompanied by an increase in rates of calcium release and reuptake, along with a significant increase in sarcoendoplasmic reticulum ATPase expression. (Yant, et al). Various combinations of insulin, glucocorticoids (such as dexamethasone) and the phosphodiesterase inhibitor 3-isobutyl-1-methil-xanthine have been shown to improve CM maturity in culture, but it is unlikely that medium additives in themselves will induce complete CM maturation (Denning).

### Example 1: Novel Single Gene Metabolic Reprogramming

A deep neural network approach was used to compare hundreds of transcriptomes from embryonic, fetal and adult cells including the PureStem® cell bank of hundreds of proprietary clonal embryonic progenitor cell lines (West). This led to the discovery of COX7A1 as a marker of the embryonic to fetal transition. COX7A1 is not expressed during embryogenesis and is first expressed at the start of fetal development. Its expression increases through early adulthood and is maintained into old age.

COX7A1 knockout mice were created and resulted in the discovery of a glycolytic shift in heart cells from the knock-out compared to control mice. These data are consistent with repression of COX7A1 in highly glycolytic human embryonic stem cells (Shyh-Chang) and cancer cell lines (Warburg effect supporting anabolic growth) (Robinson). The metabolic impact of constitutive expression of COX7A1 was studied in two transfected cell lines, 4D20.8 (an embryonic progenitor cell line) and HT1080 (a cancer cell line) showed that the expression did indeed significantly improve the use of fatty-acid oxidation for metabolic function in these cells.

### Example 2: Novel Use of COX7A1 Overexpression to Metabolically Mature CM

CRISPR/Cas9 was employed to knock-in a tetracycline-inducible COX7A1 expression cassette into a safe-harbor locus for use in this project. This was a novel use of COX7A1 expression to push hESC-derived CM into a more mature metabolic phenotype. The use of this technology will result in minimal disruption of the expansion or cardiomyocyte differentiation potential of ESI-017. Safe harbor loci, such as AAVS1, represent genomic positions where integration of a transgene does not disrupt the expression of adjacent genes on the chromosome, and positions at which transgenes can be efficiently and accurately integrated using available methods, such as CRISPR (Pellenz). The lack of disruption of normal gene expression is important, as differentiation of hES cells into target cell types involves the sequential activation (and suppression) of large numbers of gene networks. Older methods of random gene integration run the risk of disrupting genes at unintended loci, leading to altered expression and gene mutations.

The hPSC-CM product according to the invention displays a more mature metabolic phenotype for use drug safety testing and basic research. CX7-ESI-017 CM overexpresses COX7A1 resulting in a more mature metabolic phenotype in assays for cardiotoxicity testing. The assay features metabolically mature populations of CM as measured by their utilization of fatty acids as an energy source as opposed to existing hPSC-derived CM products that primarily utilize glucose as an energy source. The CX7-ESI-017 CM is economic and reproducible to manufacture. This method can further produce CM products derived from other hPSC lines, including induced pluripotent stem cells (iPSC), allowing for access to hPSC-CM with various genetic profiles.

The invention provides a scalable production method to produce populations of metabolically-mature CM derived from the transfected hES line ESI-017 expressing COX7A1.

### Example 3: Development and Validation of COX7A1 Inducible Donor Plasmid

A conditional expression cell line is developed according to the invention using CRISPR/Cas9. Transfection conditions for the ESI-017 cell line have been optimized by transfection with GFP-expressing plasmid under three electroporation conditions using a Neon system. An optimal 10-15% transfection efficiency was achieved at 1200V, 30 ms, 1 pulse.

A range of drug concentrations (puromycin) was tested for conducting effective transient drug selection after transfection. 0.3 ug/ml was selected for experiments. The complete genome sequence of the ESI-017 line was used to evaluate guide RNA sequences for a safe harbor loci integration. A CRISPR/Cas9 plasmid vector system was constructed in which the COX7A1 coding sequence is expressed from a promoter utilizing the tetracycline-inducible activator (TET-ON system). Donor plasmid assembly was evaluated by complete DNA sequence of the construct.

Validation of the expression of the COX7A1 transcript and protein is assessed first by transient transfection of the donor plasmid into HEK293 cells using RT-PCR and Western blot analysis respectively. Induction of the COX7A1 cassette is evaluated using a range of inducer (tetracycline) concentrations. Donor plasmid transfected cells express at least 10× more COX7A1 protein than mock-transfected controls.

### Example 4: Transfection, Selection and Sequence Confirmation of ESI-017

Feeder-free cultures of ESI-017 are transfected using the optimized conditions and selective agent concentrations as described above. Transfected cells are cultured at limiting dilution and placed under selection. Drug-resistant colonies are expanded and tested by PCR for integration of the donor plasmid sequence at the targeted safe harbor loci. At this stage, PCR positive clones contain either one or two copies of an integrated plasmid at the safe harbor locus.

Individual clonal lines are expanded using feeder free cell culture and then thoroughly evaluated for accurate integration at the safe harbor locus by DNA sequence analysis. Clonal lines that contain two appropriately integrated copies of the donor plasmid are prioritized for evaluation, expanded using feeder free culture and cryo-preserved for further study.

### Example 5: Testing of the COX7A1 Expression and Expansion Capacity of CX7-ESI-017 Clonal Cell Lines

Clonal cell lines and the Parental ESI-017 line are expanded in culture for at least nine population doublings and cryopreserved for further testing. The expression of the COX7A1 transgene is assessed in uninduced cultures (testing “leakiness” of the transgene) and with a range of inducer (tetracycline) concentrations using Western blot analysis.

Utilizing a CRISPR-directed transgene with expression controlled by an inducible promoter can be important for achieving the desired control of COX7A1 protein expression and will allow control of both the timing and amplitude of the transgene expression. This capability can impact the differentiation and maturation of CX-ESI-017 to cardiomyocytes and therefore this system is designed to achieve optimal control of the transgene induction.

The use of CRISPR-mediated DNA modification will result in accurate, targeted gene modification which limits the likelihood of unintended, offsite mutation. CRISPR technology is now the preferred method of chromosome modification and is well established. The use of an inducible gene expression system accurately controls the timing of the expression of the COX7A1 transgene. If the inducibility of the expression is difficult to obtain, or if deemed unnecessary, a constitutive expression vector system can be introduced by CRIPSR using the same procedure. CRISPR/Cas9 can precisely place the cassette in a safe harbor, thereby minimizing the chance that the CX7-ESI-017 expansion potential will be affected relative to Parental ESI-017, or that the CM differentiation potential of CX7-ESI-017 to CM will be affected.

Genome integration of the transgene is confirmed by PCR.

Clones are tested for COX7A1 protein expression and maximal expression based on tetracycline dose response is determined using Western blot analysis.

It is further confirmed that integration of the COX7A1 transgene into ESI-017 did not affect the line's ability to successfully differentiate into cardiomyocytes

Parental ESI-017 and CX7-ESI-017 lines are differentiated to cardiomyocytes using a modification of the method described by Burridge et al, which is referred to as the Wnt pathway Cardio-differentiation. In brief, cells are plated onto Matrigel in Essential 8 medium (Gibco). The medium is changed to RPMI 1640 supplemented with B-27(no insulin) (Gibco) containing the GSK3 inhibitor CHIR99021. After two days the media is changed to RPMI 1640 supplemented with B-27(no insulin) supplemented with a Wnt-inhibitor. Within six days of addition of a Wnt-inhibitor cells begin spontaneously contracting as a syncytium. Once the cells have started beating they may be further purified to >90% cardiomyocytes by selection for lactate dehydrogenase (preferentially expressed in cardiomyocytes) by culturing in glucose free, lactate supplemented RPMI 1640 medium for three days.

The Wnt pathway cardio-differentiation is a robust process and is widely used for differentiation of cardiomyocytes from iPSCs and hESC. The method yields >90% cardiac troponin T (cTnT) expressing cells when paired with lactate selection/glucose depletion and there are several points in the protocol that may be optimized to specific clonally derived or genetically distinct cell lines to ensure high quality and reproducible yield of Cardiomyocytes. These points include determining optimal ranges in cell plating densities, concentrations of GSK3 inhibitor, and testing different Wnt-inhibitor molecules. These clonal line(s) are then selected based on capacity to differentiate to Cardiomyocytes (>70% cTnT) in the absence of lactate selection.

### Example 6: Testing Differentiated Cultures of CM Derived from ESI-017 and from CX7-ESI-017 (CX7-ESI-017 CM) for the Expression of Markers of CM

The Parental ESI-017 line and clonal CX7-ESI-017 lines are differentiated using the cardiomyocyte differentiation process described above. Differentiated cardiomyocyte cells are lifted via enzymatic disaggregation and cryopreserved (frozen) for analysis and further use. Cardiomyocytes are characterized by flow cytometry using a prioritized panel of antibodies to establish a baseline for sarcomeric proteins (CSQ, PLN, RYR2, and SERCA/ATP2A2), contractile proteins, and receptors (MYH7, MYH6, TNNI3, TNNI1, and ADRA1A).

The clonal CX7-ESI-017 lines retain the ability to differentiate to cardiomyocytes as compared to the Parental ESI-017 line, confirming that clonal selection for introduction of the transgene and clonal expansion has not altered their differentiation capacity.

CX7-ESI-017 CM preferably express at least 70% cTnT by flow cytometry without lactate purification and CM markers at levels within 20% of the Parental ESI-017 line under the same conditions.

Cardiomyocytes spontaneously beat with phase-contrast microscopic examination. Cardiomyocytes are characterized by flow cytometry for a panel of the sarcomeric proteins including CSQ, PLN, RYR2, and SERCA/ATP2A2.

Cardiomyocytes are positive for the expression of the genes for the following panel of contractile proteins and receptors: MYH7, MYH6, TNNI3, TNNI1, and ADRA1A.

### Example 7: Optimizing the Timing of Induction and Inducer Dose Response Relative to CM Differentiation

CX7-ESI-017 lines and the Parental ESI-017 line are subject to the cardiomyocyte differentiation protocol (using expanded CX7-ESC-017 cultures). During this protocol, cultures are treated with two dose levels of tetracycline at four different time-points during the differentiation process: i) Concurrent with initiation of differentiation; ii) Concurrent with the addition of Wnt inhibitor; iii) upon the first observation of spontaneous contraction; and iv) simultaneously with lactate selection. Once the cells have been differentiated for at least 14 days as described above, the cells are lifted via enzymatic disaggregation and cryopreserved for analysis and further use. The expression of COX7A1 protein is confirmed with Western blot analysis for each culture. Induction conditions result in robust expression of COX7A1 after 14 days of differentiation.

### Example 8: Characterization of CM Differentiation with COX7A1 Induction

Cryopreserved cultures identified and selected for robust expression of COX7A1 are thawed, plated, cultured and assessed for CM differentiation. Cells are characterized by flow cytometry for a panel of markers to establish a baseline for further study including testing by flow cytometry for a panel of sarcomeric proteins (CSQ, PLN, RYR2, and SERCA/ATP2A2) and contractile proteins and receptors (MYH7, MYH6, TNNI3, TNNI1, and ADRA1A). The tetracycline dose/timing that induces the highest expression levels of COX7A1 is prioritized for testing and compared to uninduced (no tetracycline) differentiated cells and cardiomyocytes differentiated from Parental ESI-017 cells.

The ESC differentiated to cardiomyocytes are tested for metabolic maturity as measured by preferential utilization of fatty acids over glucose as an energy source: This task is performed by Pluristyx Inc, using a Seahorse XF96 metabolism analyzer. CX7-ESI-017 CM from one clonal transgenic sub-line identified earlier and CM from the Parental ESI-017 is thawed and re-plated to 96 well “fluxpak” plates for assay in the Seahorse system. The CM on fluxpak plates is then tested for glucose utilization by real-time measurements of oxygen consumption rate (OCR) and extracellular acidification rate (ECAR) in live cells. OCR measurements are directly related to cellular respiration (after removing contribution from non-mitochondrial oxygen consumption by injection of mitochondrial inhibitors). CM on fluxpak plates are also tested for Fatty Acid Oxidation (FAO) XF Palmitate-BSA FAO Substrate, the Agilent Seahorse XF Cell Mito Stress Test Kit, and Etomoxir (Eto), a carnitine palmitoyl transferase-1 inhibitor. The XF Analyzer allows for the simultaneous measurement of the two major energy pathways of the cell: mitochondrial respiration and glycolysis. Testing of mitochondrial location and density is then be tracked by tetramethylrhodamine (TMRE) staining via epi-fluorescent microscopy.

Properly timed and dosed transgenic expression of COX7A1 by drug inducible promoter results in metabolic and phenotypic maturation of cardiomyocytes as compared to cardiomyocytes differentiated from uninduced CX7-ESI-017 or control ESI-017. Maturation is demonstrated by: i) decreased glycolytic metabolism and increased fatty acid metabolism relative to controls and; ii) increased expression of sarcomeric proteins, the receptor ADRA1A, and the adult contractile proteins MYH7 and TNNI3 as compared to control cells. The timing of induced transgenic expression of COX7A1 has a quantifiable impact on both differentiation and maturation of hES-derived CM cells. Differentiation efficiency is assessed as the percentage of cells expressing cTnT. Metabolic maturation of Cardiomyocytes is measured by comparing mitochondrial density and location using TMRE staining and determining the metabolic function by determining levels of glycolysis, mitochondrial respiration, and fatty-acid oxidation.

The use of CRISPR-mediated insertion of transgenic drug inducible genes is a commonly used genetic editing tool for pluripotent stem cells. Similarly, the Seahorse XF96 metabolism analyzer is a well-established tool to understand the metabolism of cultured mammalian cells.

Cells induced to express the COX7A1 transgene produce COX7A1 protein and differentiate to cardiomyocytes. Induction of COX7A1 at different tetracycline doses and time points may result in different efficiencies of cardiomyocyte differentiation. Cardiomyocytes with COX7A1 expression exhibit a more mature phenotype measured by increased expression of sarcomeric proteins, adult contractile proteins, and the receptor ADRA1A. Cardiomyocytes also have more and larger mitochondria with a more dispersed arrangement (as opposed to a more peri-nuclear arrangement) and cells demonstrate a metabolic switch from reduced glycolysis to increased fatty acid utilization.

COX7A1 expressing cardiomyocytes from at least one CX7-ESI-017 clonal line express at least 70% cTnT by flow cytometry without lactate purification.

COX7A1 expressing cardiomyocytes beat at a significantly slower pace than uninduced or control cells and may stop spontaneously contracting as measured by phase-contrast microscopic examination.

COX7A1 expressing cardiomyocytes express more (increased percentage of cells and higher mean fluorescent intensity) of one or more of the sarcomeric proteins CSQ, PLN, RYR2, and SERCA/ATP2A2 as measured by flow cytometry.

COX7A1 expressing cardiomyocytes express one or more (increased percentage of cells and higher mean fluorescent intensity) of mature CM contractile proteins and receptors: MYH7, TNNI3, and ADRA1A.

COX7A1 expressing Cardiomyocytes demonstrate increased mitochondrial respiration and reduced glycolysis as compared to uninduced cardiomyocytes or Parental ESI-017 derived cardiomyocytes. COX7A1 expressing cardiomyocytes have more mitochondria distributed across the cell and the mitochondrial stain more intensely as tracked by TMRE staining via epi-fluorescent microscopy than uninduced cardiomyocytes or parental ESI-017 derived Cardiomyocytes.

Cardiomyocytes derived from induced cultures express a more mature metabolic phenotype by an enhancement in the utilization of fatty acid oxidation and a reduction in glycolysis. The cardiomyocytes also have a more mature sarcomeric phenotype with an increased expression of CSQ, PLN, RYR2, and SERCA/ATP2A2 and express substantially more of the genes MYH7, TNNI3, and ADRA1A and less MYH6 and TNNI1 than in uninduced cells. Induced cells will beat slower, if at all, as compared to uninduced cells indicating cellular maturation driven by COX7A1 expression

### Example 9: Assessment of CX-ESI-017 CM as a Superior Model of Detecting Cardiotoxicity

Cardiomyocytes derived from un-induced CX7-ESI-017 or Parental ESI-017 will register less sensitivity to the cytotoxic effects of doxorubicin and cytarabine, two chemotherapeutic agents with known cardiotoxicity, relative to induced CX7-ESI-017-CM.

Cardiomyocytes toxicity is assessed via confocal imaging of Annexin-V staining (apoptosis) and propidium iodide incorporation (late stage apoptosis or necrosis) (Hawkins) Camptothecin and the pan-caspase inhibitor Z-VAD-FMK is used as positive and negative controls, respectively. The caspase 9 inhibitor Z-LEHD-FMK is used to assess the mitochondrial initiation of apoptosis.

Induced and non-induced CX7-ESI-017-CM are incubated with combinations of dichloroacetate and etomoxir and then challenged by cardiotoxic doses of doxorubicin and cytarabine. Mechanistically, dichloroacetate stimulates pyruvate dehydrogenase and glycolysis (Stacpoole) and shifts mitochondrial metabolism to glycolysis. Etomoxir blocks mitochondrial fatty acid uptake and oxidation by inhibiting the activity of carnitine palmitoyltransferase I. Camptothecin, Z-VAD-FMK, and Z-LEHD-FMK will serve as controls as in 4.A. Cardiotoxicity is assessed via confocal imaging of Annexin-V staining (apoptosis) and propidium iodide incorporation (late stage apoptosis or necrosis). Induced CX7-ESI-017-CM exhibit increased sensitivity to both doxorubicin and cytarabine following a shift in mitochondrial metabolism from fatty acid oxidation to glycolysis (dichloroacetate+etomoxir). Change in cardiotoxicity in non-induced CX7-ESI-017-CM under the same conditions is not expected.

CX7-ESI-017 CM express a higher level of maturity relative to beating rate, cardiomyocyte markers and metabolic maturity. Cardiomyocytes beat at a slower pace than uninduced or control cells (range of 20-25 percent slower) and may stop spontaneously contracting as measured by phase-contrast microscopic examination.

Cardiomyocytes express more (20 percent higher mean fluorescent intensity) of the sarcomeric proteins CSQ, PLN, RYR2, and SERCA/ATP2A2 as measured by flow cytometry.

Cardiomyocytes express more (20 percent higher mean fluorescent intensity) of mature CM contractile proteins and receptors: MYH7, TNNI3, and ADRA1A.

Cardiomyocytes demonstrate 15-20 percent increased mitochondrial respiration and a 5-10 percent reduction in glycolysis as compared to uninduced CX7-ESI-017 CM or Parental ESI-017 derived CM. CX7-ESI-017 CM have approximately 5-10 percent more mitochondria distributed across the cell and the mitochondrial stain approximately 5-10 percent more intensely as tracked by TMRE staining via epi-fluorescent microscopy than uninduced cardiomyocytes or parental ESI-017 derived cardiomyocytes.

The phenotypic immaturity of hPSC-derived CM is a detriment for their use in in vitro models to test the potential of a drug to have cardiotoxic effects (Denning, Bernstein). Maturation of CM metabolism occurs when CM are exposed to higher demands of energy as well as oxygen and fatty acids and a switch to a high metabolic stress may be one of the driving forces of maturation (Gaetanol). Disruption of the respiratory chain function has been shown to prevent mitochondrial organization and compromise the energetic infrastructure, causing deficient sarcomerogenesis and contractile malfunction (Chung).

Therefore, a switch to a higher metabolic stress (as seen by increased use of fatty acid metabolism) will lead to maturation of the CM's calcium kinetics, (as reflected by a higher level of sarcomeric proteins CSQ, PLN, RYR2, and SERCA/ATP2A2) and the maturation of CM contractile machinery (as seen by higher levels of MYH7, TNNI3 and ADRA1A).

Human induced pluripotent stem cell derived CM have detect doxorubicin-induced cardiotoxicity (Burridge (2016), Louisse) yet the lack of suitable maturity has been listed as a weakness in these models (Burridge (2016)).

### Example 10: Micropatterned Co-Cultured Cardiotoxicity Assay

The invention is further directed to a micropatterned co-cultured human-pluripotent cell-based in-vitro cardiotoxicity assay provided in a shippable multi-well format for drug safety testing. Micropatterning and co-culture has been shown in the literature (see references below) to be feasible and to further improve the phenotypic maturity of hPSC-derived cardiomyocytes. In addition, cardiomyocytes derived from hPSC are generally a mixed cell population and this cellular heterogeneity could adversely skew results in studies trying to model either atrial or ventricular arrhythmias (Bernstein). The purification and isolation of ventricular CM (VCM) and nodal “pacemaker” cardiomyocytes (NCM) is also shown in the literature to be feasible (see references below). The assay according to the invention features cells that display characteristics of mature cardiac tissues that can be economically and reproducibly manufactured. Each well of the assay contains purified compartmentally-isolated populations of mature ventricular cardiomyocytes (VCM) co-cultured with vascular endothelial cells and pericytes and compartmentally-isolated nodal cardiomyocytes (NCM) (pacemaker cells) that will drive the VCM action potential and beat rate.

This technology has been shown in published literature to improve the maturation level and function of both primary and hPSC-derived hepatocytes (March, Berger).

Co-culture of metabolically-mature ESI-017 CM with endothelial cells and pericytes derived from ESI-017 to enhance CM maturity. Cardiomyocytes, fibroblasts and vascular cells (endothelial cells, vascular smooth muscle cells and pericytes) constitute the primary cell types of the adult mammalian heat (Dobaczewski). Co-culture PSC-derived CM with endothelial cells and/or pericytes has been shown in the literature to improve hPSC-CM maturity and function (Lee, Masumoto).

The invention includes a highly scalable system to produce endothelial progenitors and pericytes from ESI-017 (Greenwood-Goodwin). The co-culture enhances and optimizes the structural and functional of the ESI-017 derived cardiomyocytes.

### Example 11: Derivation and Purification of Ventricular CM (VCM) and Nodal (Pacemaker) CM (NCM) from ESI-017

Techniques to create pure populations of cardiomyocytes sub-types exist in the field. Modulation of retinoic acid signaling during hESC differentiation is used to generate atrial and ventricular like cardiomyocytes (Devella). A NKX2.5-GFP targeted hESC line is further transfected with an inducible MHY (myosin heavy chain) construct and using insulin-like growth factor-1 and a hedgehog pathway agonist, cardiac progenitor cells can be isolated and propagated to over 40 populations doublings. Through modulation of exogenous BMP, FGF, Wnt and RA signaling these populations are differentiated down pathways leading to ventricular or nodal Cardiomyocytes (Birket). The invention further includes scalable purified populations of VCM and NCM from ESI-017.

### Example 12: Micropattern Well

A structured elastomeric stamp provides a relief pattern (micropatterned extracellular matrix) in the well of a 96-well culture plate that allows a physiologically relevant plated cardio-tissue architecture. As observed with in vitro hepatocyte models, published literature describing in vitro cardiomyocyte models that control the spatial orientation of cardiomyocytes (as opposed to the chaotic environment of cardiomyocyte plated randomly on a non-patterned surface) show improved maturity and function. VCMs are oriented parallel to each other along their long axis. This gives them a property called anisotropy (having a physical property that has a different value when measured in different directions). In the case of cardiomyocytes, their contractile force is directed along their long axis. Having the proper anisotropic orientation has been shown to improve hPSC-CM maturity (Pong, Rao). There is a need for a model that isolates nodal pacemaker cells from VCM and presents the two populations in a cellular architecture that more closely mimics that found in the heart. Unlike other cardiomyocytes, pacemaker cells do not require external stimulation to initiate their action potential; they are capable of self-initiated depolarization in a rhythmic fashion, a property known as automaticity. In vivo the sinoatrial (SA) node contains the NCN that set the heat's beat rate. The nodal pacemaker cells are arranged in a complex and non-uniform tissue with unique ion channels and cell-cell junctions and are physically shielded from the hyperpolarizing influence of the atrial muscle yet can drive the atrial muscle (Boyett, Murphy).

The micropatterned surface pattern of parallel rows or furrows aligns the VCM along their long axis (shown to allow anisotropy) and the proper placement of cell-cell and cell-matrix junctions. A separated micropatterned compartment is placed the nodal cells in a shielded location apart, but nominally connected, to the patterned VCM. This arrangement improves the metabolic maturity of the VCM as reflected in a more “rod shaped” morphology and organized sarcomeres (˜2.2 microns in length) with the display of Z-discs, I, H, A and M bands and t tubules. Structural maturity will be reflected in VCM alignment along their long axis and the presence of end-to-end cell junctions in the axial direction (intercalated discs) and cell-to-matrix junctions (costameres) in the lateral direction. The cells will form a syncytium that will exhibit anisotropy. The VCM is paced through a nominal connection window to a purified population of compartmentally-isolated nodal pacemaker cells micropatterned on their unique extracellular matrix. See FIG. 5.

### Example 13—Lentiviral Vector for hCOX7A1 Expression

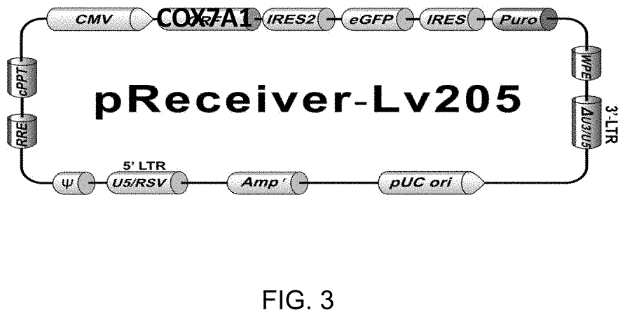

A cDNA encoding the human COX7A1 mRNA was cloned into a lentiviral expression vector. See FIG. 3. Expression of the COX7A1 transcript is driven by the cytomegalovirus early promoter (CMV) and a puromycin-resistance gene allows for selection of transgenic cells using medium containing puromycin.

### Example 14—Anti-COX7A1 Western Blot

Protein lysates were prepared from HT1080 human fibrosarcoma cells or from the HT1080 cells transduced with a lentiviral preparation that encodes a CMV-driven hCOX7A1 transgene.

Lysates were resolved by SDS-PAGE and transferred to a membrane by Western blotting.

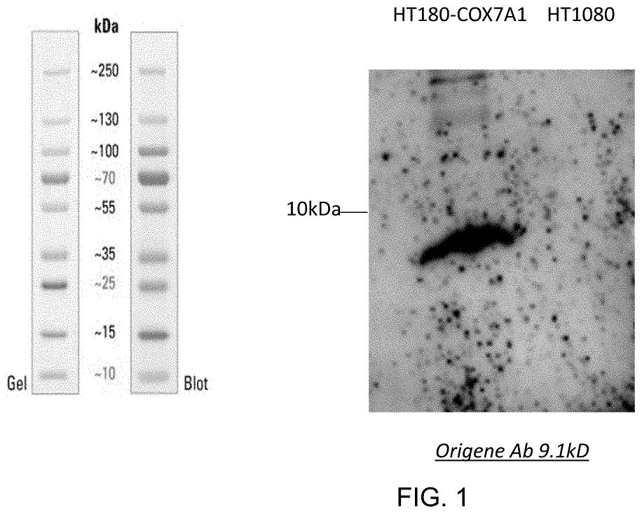

The Western blot was exposed to a rabbit anti-hCOX7A1 antisera and then a goat anti-rabbit antisera, followed by luminescence detection. See FIG. 1.

### Example 15—COX7A1 RT-PCT Expression in Stable Cell Lines

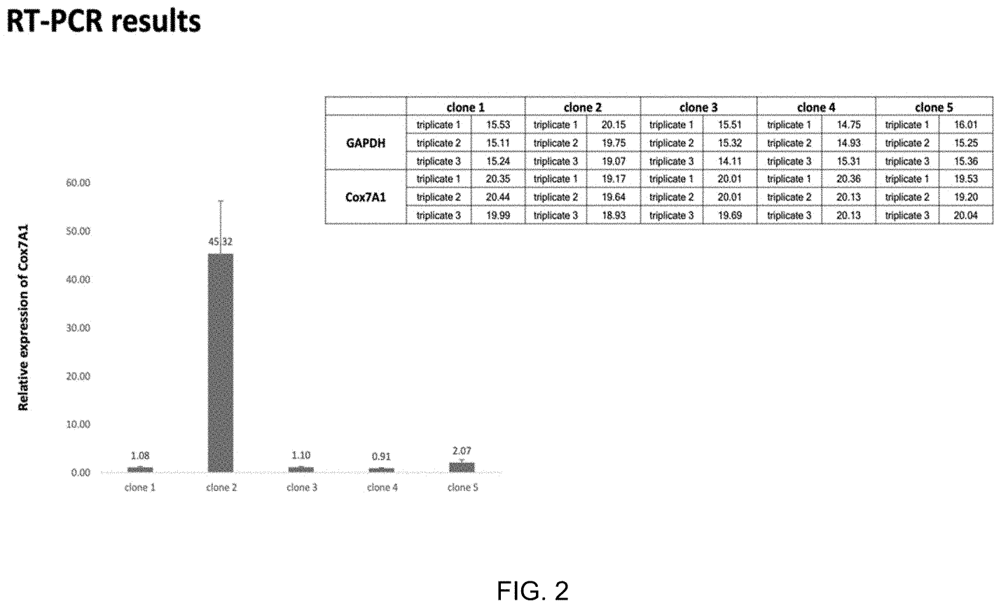

Human ESI017 embryonic cells were transduced with a lentivirus preparation and individual clonal lines were selected using puromycin. Nucleic acid lysates were prepared the clones and cDNA was prepared using reverse transcriptase. The resulting cDNA was analyzed by Q-PCR methods using transgene-specific PR primers. The resulting signal was quantified by comparison to the endogenous house-keeping mRNA for glyceraldehyde dehydrogenase (GPADH). See FIG. 2.

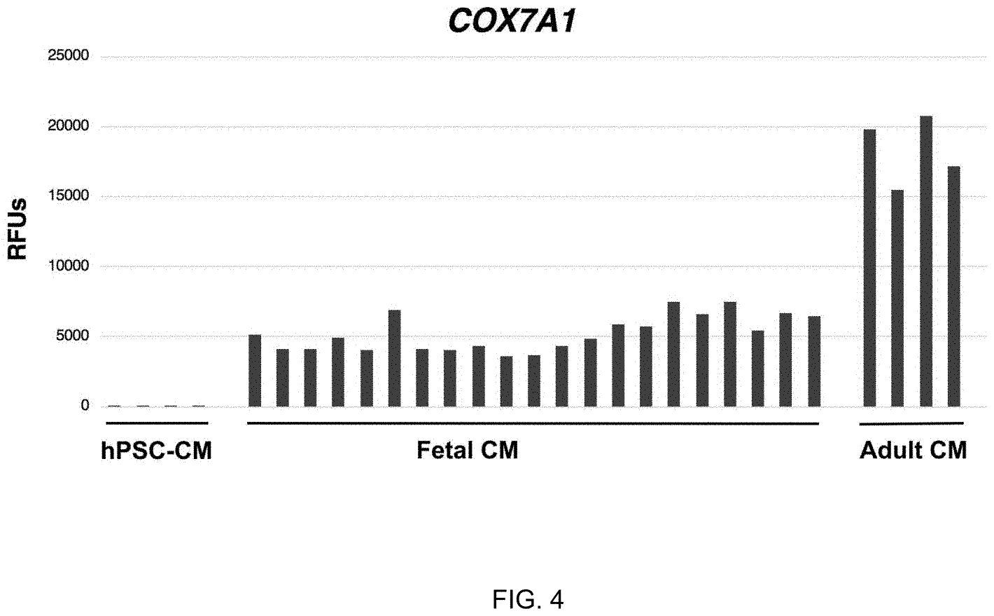

As shown in FIG. 4, as determined by Illumina bead array gene expression analysis, gene expression (shown in relative fluorescence units) is essentially undetectable in iPSC-CM, then increases in expression during fetal development in vivo, then increases further upon reaching adulthood. The exogenous expression of COX7A1 in hPSC-CM is preferably within 10-fold of the expression of adult CM as shown, most preferably within 2-fold of the adult CM expression.

Although the description herein contains many details, these should not be construed as limiting the scope of the disclosure but as merely providing illustrations of some of the presently preferred embodiments. Therefore, it will be appreciated that the scope of the disclosure fully encompasses other embodiments which may become obvious to those skilled in the art.

In the claims, reference to an element in the singular is not intended to mean “one and only one” unless explicitly so stated, but rather “one or more.” All structural, chemical, and functional equivalents to the elements of the disclosed embodiments that are known to those of ordinary skill in the art are expressly incorporated herein by reference and are intended to be encompassed by the present claims. Furthermore, no element, component, or method step in the present disclosure is intended to be dedicated to the public regardless of whether the element, component, or method step is explicitly recited in the claims. No claim element herein is to be construed as a “means plus function” element unless the element is expressly recited using the phrase “means for”. No claim element herein is to be construed as a “step plus function” element unless the element is expressly recited using the phrase “step for”.

### REFERENCES

Magdy, T. et al., Human Induced Pluripotent Stem Cell (hiPSC)-Derived Cells to Assess Drug Cardiotoxicity: Opportunities and Problems. Annual Review of Pharmacology and Toxicology. 2018; 58:83-103.
Siramshetty, B. V. et al., WITHERAWN—a resource for withdrawn and discontinued drugs. Nucleic Acids Research. 2016; 44: database issue doi: 10.1093/nar/gkv1192
Gintant, G. et al., Evolution of strategies to improve preclinical cardiac safety testing. Nat. Rev. Drug Discov. 2016; 15:457-71.
Denning, C. et al., Cardiomyocytes from human pluripotent seem cells: From laboratory curiosity to industrial biomedical platform. Biochimica et Biophysica Acta. 2016; 1863 1728-1748.
Chung, S. et al., Mitochondrial oxidative metabolism is required for the cardiac differentiation of stem cells. Nat Clin Pract Cardiovasc Med. 2007; February; 4:(Supp 1): s60-s67. Doi: 10.1038/ncpcardio0766.
Huttemann, M. et al., Mice deleted for heart-type cytochrome c oxidase subunit 7a1 develop dilated cardiomyopathy. Mitochondrion. 2012; 12:294-304.
West, D. M. et al., Use of deep neural network ensembles to identify embryonic-fetal transition markers: repression of COX7A1 in embryonic and cancer cells. 2018; Oncotarget. 2018; 9:(No. 8), 7796-7811.
Shyh-Chang N. et al., Stem cell metabolism in tissue development and aging. Development. 2013; 140:2535-47.
Robinson G. L. et al., Switching from aerobic glycolysis to oxidative phosphorylation modulates the sensitivity of mantle cell lymphoma cells to TRAIL. Oncogene. 2012; 31:4996-5006.
Pellenz S. and Monnat R. J. Identification and analysis of genomic homing endonuclease target sites. 2014; Methods Mol. Biol. 1123: 245-64.
Burridge P. W. et al., Chemically defined generation of human cardiomyocytes. Nat Methods. 2014; 11(8):855-60.
Gaetano J. et al., Naturally engineered maturation of cardiomyocytes. Front. Cell Dev. Bio. 2017; 5:50. Doi: 10.3389/fce11.2017.00050
March, S. et al., Micropatterned coculture of primary human hepatocytes and supportive cells for the study of hepatotropic pathogens. Nature Protocols 10:(12) 2027-2052 (2015).
Berger, D. R. et al., Enhancing the Functional Maturity of Induced Pluripotent Stem Cell-Derived Human Hepatocytes by Controlled Presentation of Cell-Cell Interactions In Vitro. Hepatoloty 61(4): 1370-1381 (2015)
Dogaczewski, M. et al., The extracellular matrix modulates fibroblast phenotype and function in the infarcted myocardium. J Cardiovasc Transl Res. 2012; December; 5(6): 837-847.
Lee, D. S. et al., Defined Micro RNAs induce aspects of maturation in mouse and human embryonic-stem-cell-derived cardiomyocytes. Cell Reports 2015; 12:1960-1967.
Greenwood-Goodwin M. et al., A novel lineage restricted, pericyte-like cell line isolated from human embryonic stem cells. Sci Rep 2016; 6:24403; doi:10.1038/srep24403.
Birket, M. J. et al., Expansion and patterning of cardiovascular progenitors derived from human pluripotent stem cells, Nat. Biotechnol. 2015; 33:(9) 970-979.
Devalla, H. D. et al., Atrail-like cardiomyocytes from human pluripotent stem cells are a robust preclinical model for assessing atrial-selective pharmacology. EMBO Mol. Med. 2015. 7:(4) 394-410.
Pong, T. et al., Hierarchical architecture influences calcium dynamics in engineered cardiac muscle. Exp Biol Med. 2011. March; 236(3): 366-373.
Rao, C., et al. The effect of microgrooved culture substrates on calcium cycling of cardiac myocytes derived from human induced pluripotent stem cells. Biomaterials 2013. 34:(10) 2399-2411.
Murphy, C. et al., Current concepts of anatomy and electrophysiology of the sinus node. J. Interv Card Electrophysiol. 2016; 46:9-18.
Boyett M. R. et al., The sinoatrial node, a heterogeneous pacemaker structure. Cardiovascular Res. 2000; 47:658-687.
Hawkins, B. J. et al., Superoxide flux in endothelial cells via the chloride channel-3 mediates intracellular signaling. Mol Biol Cell 18, 2002-2012; doi:10.1091/mbc.E06-09-0830 (2007).
Stacpoole, P. W. The pharmacology of dichloroacetate. Metabolism: clinical and experimental 1989; 38, 1124-1144.
Bernstein D. Induced pluripotent stem cell-derived cardiomyocytes: a platform for testing for drug cardiotoxicity. Prog Pediatr Cardiol. 2017. September; 46:2-6. Doi: 10.1016/j.ppedcard.2017.07.001.
Louisse, J., Assessment of acute and chronic toxicity of doxorubicin in human induced pluripotent stem cell-derived cardiomyocytes. Toxicol. In Vitro. 2017. August; 42:”182-190. Doi: 10.1016/j.tiv.2017.04.023.
Burridge, W B, et al., Human induced pluripotent stem cell-derived cardiomyocytes recapitulate the predilection of breast cancer patients to doxorubicin-induced cardiotoxicity. Nat Med. 2016; May, 22(5): 547-556 doi:10.1038/nm.4087.

1. A method of screening a composition for cardiotoxicity comprising contacting the composition with cardiomyocytes that have increased fatty acid oxidation and/or diminished glucose oxidation.

2. The method of claim 1, wherein the cardiomyocytes are prepared by overexpression of COX7A1.

3. The method of claim 1, wherein the cardiomyocytes are human pluripotent stem cell-derived cardiomyocytes.

4. The method of claim 1, wherein the cardiomyocytes are a derived from a human induced pluripotent stem cell line.

5. A composition comprising a cardiomyocyte or population of cardiomyocytes that primarily utilize fatty acids as an energy source, wherein the cardiomyocyte or population of cardiomyocytes is prepared by overexpression of COX7A1.

6. A method of generating a mature cardiomyocyte or population of cardiomyocytes, comprising transfecting a human pluripotent stem cell line with COX7A1, differentiating those cells to produce cardiomyocytes, and maturing the cardiomyocytes via expression of COX7A1.

7. The method of claim 6, wherein the mature cardiomyocyte or population of cardiomyocytes have increased fatty acid oxidation and/or diminished glucose oxidation relative to cardiomyocytes differentiated from a human embryonic stem cell line not transfected with COX7A1.

8. The method of claim 6, wherein the mature cardiomyocyte or population of cardiomyocytes have an increased expression in one or more of CSQ, PLN, RYR2, SERCA/ATP2A2, MyH7, TNNI3, and ADRA1A and/or a decreased expression of one or more of MYH6 and TNNI1 than cells not expressing COX7A1.

9. The method of claim 6, wherein COX7A1 is transfected by a knock-in inducible COX7A1 expression cassette.

10. The method of claim 6, wherein COX7A1 is available by constitutive expression.

11. The method of claim 1, wherein the screening is conducted in a culture vessel wherein at least one type of cardiomyocyte is on a micropatterned surface that orients the cardiomyocytes in a specific pattern.

12. The method of claim 11, wherein the cardiomyocytes on the micropatterned surface are ventricular cardiomyocytes.

13. The method of claim 11, wherein ventricular cardiomyocytes on the micropatterned surface have at least one contact point with nodal cells.

14. The method of claim 11, wherein the micropatterened surface comprises fibronectin.

15. The method of claim 11, wherein the cardiomyocytes are cocultured with at least one secondary cell capable of improving cardiomyocyte maturity.
